(19) United States
(12) Patent Application Publication
(10) Pub. No.: US 2010/0226987 A1
(43) Pub. Date: Sep. 9, 2010

(54) TARGETING CONJUGATES COMPRISING ACTIVE AGENTS ENCAPSULATED IN CYCLODEXTRIN-CONTAINING POLYMERS

(75) Inventors: Jallal M. Gnaim, Baka El Garbia (IL); Muhammad Athamna, Kfar Qari (IL)

Correspondence Address: BROWDY AND NEIMARK, P.L.L.C.;624 NINTH STREET, NW, SUITE 300, WASHINGTON, DC, 20001-5303, US

(73) Assignee: CAPSUTECH LTD., Nazareth Illit (IL)

(21) Appl. No.: 12/666,939

(22) PCT Filed: Jun. 29, 2008
(86) PCT No.: PCT/IL08/00884

§ 371 Date: May 6, 2010

Related U.S. Application Data
(60) Provisional application No. 60/946,775, filed on Jun. 28, 2007.

(51) Int. Cl. A61K 9/14; A61K 9/00; A61K 31/704; A61K 31/337; C07H 21/00; C07K 14/00; C07K 2/00; C08B 37/16; A61P 35/00

(52) U.S. Cl. 424/488; 424/400; 514/34; 514/449; 536/23.1; 530/300; 530/395; 536/103

(57) ABSTRACT

A targeting conjugate is provided comprising an active agent, one or more residues of a cyclodextrin (CD)-containing polymer and a biorecognition molecule. The polymer is preferably a peptide or a polypeptide comprising at least one amino acid residue containing a functional side group to which at least one of the CD residues is linked covalently; the biorecognition molecule is covalently bonded directly or via a spacer to the polymer backbone of the CD-containing polymer; and the active agent is noncovalently encapsulated within the cavity of the cyclodextrin residues and/or entrapped within the polymer matrix of the CD-containing polymer.

### FIELD OF THE INVENTION

The present invention relates to drug delivery and, in particular, relates to conjugates of a biorecognition molecule/target moiety with a cyclodextrin-containing polymer containing an encapsulated active agent, to methods for their preparation and uses thereof.

### BACKGROUND OF THE INVENTION

There is a continuous need for an effective system that delivers bioactive materials at the site of action, while minimizing peak-trough fluctuations. Ideally such a system would eliminate undesirable side effects and reduce dosage and frequency of administration while improving visible effects.

Many technologies are already in place, including multiple emulsions, microemulsions, microspheres, nano-spheres, microsponges, liposomes, cyclodextrins, skin patches and unit dosages.

Microencapsulation is a growing field that is finding application in many technological disciplines, such as in the food, pharmaceutical, cosmetic, consumer and personal care products, agriculture, veterinary medicine, industrial chemicals, biotechnology, biomedical and sensor industries. A wide range of core materials has been encapsulated. These include adhesives, agrochemicals, catalysts, living cells, flavor oils, pharmaceuticals, vitamins, and water. There are many advantages to microencapsulation. Liquids can be handled as solids; odor or taste can be effectively masked in a food product; core substances can be protected from the deleterious effects of the surrounding environment; toxic materials can be safely handled; and drug delivery can be controlled and targeted. However, the microencapsulation technology has limited use for drug targeting and poor water solubility.

Encapsulation also can occur on a molecular level. This can be accomplished, for example, by using a category of carbohydrates called cyclodextrins (CDs). Encapsulates made with these molecules may possibly hold the key for many future encapsulated formulation solutions. CDs are a general class of molecules composed of glucose units connected by α-1,4 glycosidic linkages to form a series of oligosaccharide rings. In nature, the enzymatic digestion of starch by CD glycosyltransferase (CGTase) produces a mixture of CDs comprised of 6, 7 and 8 glucose units, known as α-, β- and γ-CD, respectively, depicted below.

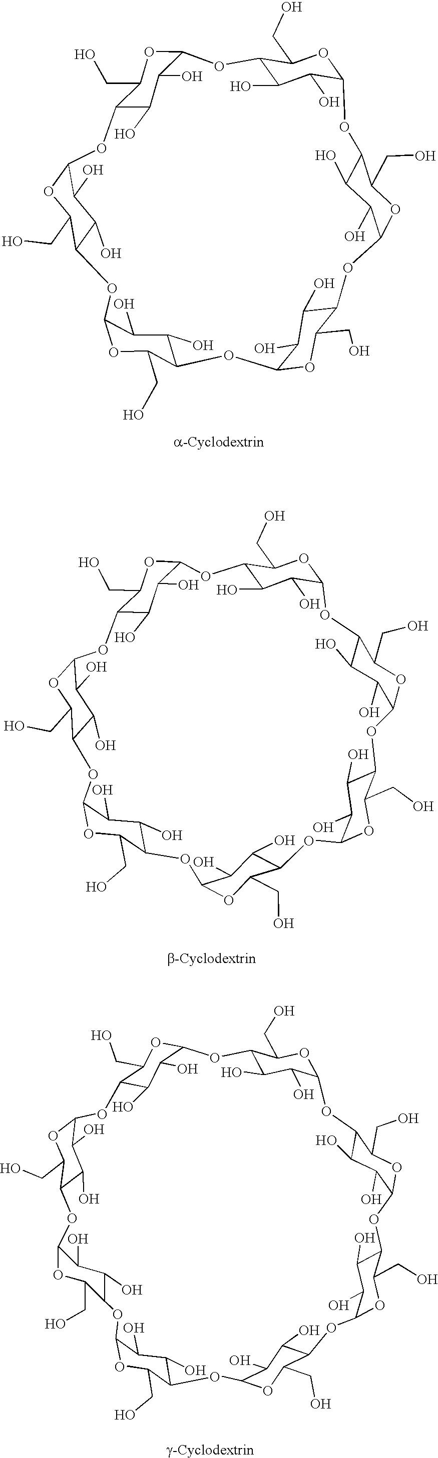

Commercially, cyclodextrins are still produced from starch, but more specific enzymes are used to selectively produce consistently pure α-, β- or γ-CD, as desired. All three cyclodextrins are thermally stable (<200° C.), biocompatible, exhibit good flow properties and handling characteristics and are very stable in alkaline (pH<14) and acidic solutions (ph>3).

As a result of their molecular structure and shape, the cyclodextrins possess a unique ability to act as molecular containers (molecular capsules) by entrapping guest molecules in their internal cavity. The ability of a cyclodextrin to form an inclusion complex with a guest molecule is a function of two key factors. The first is steric and depends on the relative size of the cyclodextrin to the size of the guest molecule. The second critical factor is the thermodynamic interactions between the different components of the system (cyclodextrin, guest, solvent). The resulting inclusion complexes offer a number of potential advantages in cosmetic and pharmaceutical formulations.

Molecular encapsulation is more comprehensive and much more controlled. For concentrated ingredients, this ability helps to assure an even dispersion in the final product. This control also helps saving on costly ingredients.

Shaped like a lampshade, the cyclodextrin molecule has a cavity in the middle that has a low polarity (hydrophobic cavity), while the outside has a high polarity (hydrophilic exterior). Since water is polar, cyclodextrin dissolves well in it. Forming a cyclodextrin complex can be as simple as mixing the cargo into a water solution of CD and then drawing off the water by evaporation or freeze-drying. The complex is so easily formed because the hydrophobic interior of the CD drives out the water through thermodynamic forces. The hydrophobic portions of the cargo molecule readily take the water's place.

As a result of their unique ability to form inclusion complexes, CDs provide a number of benefits in cosmetic and pharmaceutical formulations: bioavailability enhancement; active stabilization; odor or taste masking; compatibility improvement; material handling benefits; and irritation reduction. CDs have been used in Europe and Japan for many products (Duchene, 1987). Japanese manufacturers, in particular, have used them in many products during the past 15 years. In the United States, CD is used to remove the cholesterol from eggs (Li and Liu, 2003; Barse et al. 2003).

However, molecular encapsulation technology employing CDs suffers from several drawbacks such as limited capacity of the CD cavity, rapid release of the encapsulated active molecules under physiological conditions and low water solubility of the native β-CD. Therefore, there is still a strong need for a new class of materials which have combined advantages of both methods, namely, microencapsulation and molecular encapsulation and can target a drug to a desired target site.

U.S. Pat. No. 5,631,244 discloses a mono-6-amino-6-deoxy-β-CD derivative substituted in the 6-position by an α-amino acid residue and cosmetic or dermatological compositions comprising said CD derivative or an inclusion complex of said CD derivative and an active substance.

In the International Application PCT/IL2006/001459 published as WO 2007/072481 on Jun. 28, 2007, incorporated herewith in its entirety by reference as if fully disclosed herein, the present inventors have disclosed a modification of the known cyclodextrin-based encapsulation technology by providing a cyclodextrin (CD)-containing polymer comprising one or more CD residues, wherein said polymer is selected from a peptide, a polypeptide, a protein, an oligonucleotide, a polynucleotide or a combination thereof, and the peptide or protein comprises at least one amino acid residue containing a functional side group and at least one of the CD residues is linked to said functional side group of the peptide or protein or to the sugar moiety of the oligonucleotide or polynucleotide, and wherein an active agent is encapsulated within the cavity of said CD residues and/or is embedded within the polymer matrix. This technology enables broader and more focused applications of the CD encapsulation technique.

U.S. Pat. No. 5,068,227 discloses cyclodextrins as carriers for active agents in combination with biospecific molecules such as proteins covalently bound to the cyclodextrins. The biospecific molecules facilitate delivery of the active agents to particular sites recognized by the biospecific molecules.

### SUMMARY OF THE INVENTION

In accordance with the present invention, a biorecognition molecule is covalently coupled to the polymer backbone of the CD-containing polymer of the above-described WO 2007/072481, thus facilitating the delivery of the active agent to a biospecific target site.

The present invention thus relates to an active agent-cyclodextrin-biorecognition molecule conjugate, wherein: (i) said cyclodextrin (CD) is a CD-containing polymer comprising one or more CD residues, said polymer is selected from a peptide, a polypeptide, an oligonucleotide or a polynucleotide, the peptide or polypeptide comprises at least one amino acid residue containing a functional side group and at least one of the CD residues is linked covalently to said functional side group or to the sugar moiety of a nucleotide residue of said oligonucleotide or polynucleotide; (ii) said biorecognition molecule is covalently bonded directly or via a spacer to the polymer backbone of the CD-containing polymer; and (iii) said active agent is noncovalently encapsulated within the cavity of the cyclodextrin residues and/or entrapped within the polymer matrix of the CD-polymer.

The present invention further provides the biorecognition molecule-CD-containing polymer compounds wherein the biorecognition molecules are covalently linked either directly or via a spacer to the end group of the polymer backbone. These compounds are useful as carriers or delivery systems of active agents/drugs to the target sites recognized by the biorecognition molecules.

The present invention still further provides pharmaceutical compositions comprising the conjugates of the invention.

The conjugates of the instant invention have high water solubility and overcome the problem of low carrying capacity of individual cyclodextrins.

### BRIEF DESCRIPTION OF THE FIGURE

FIGS. 1A-1B are pictures of fluorescence microscopy showing the fluorescence associated with folate-receptor over expressing KB cancer cells, which were incubated with a mixture of the di-glutamic acid-CD, the fluorescent rhodamine-B (RhB), the biorecognition molecule folic acid (FA) and PEG, each at a concentration of 1.0 mM (control, 1A), or with the conjugate 55 (FA-PEG-CD(Glu-Glu)-encapsulated RhB) (1B).

### DETAILED DESCRIPTION OF THE INVENTION

The delivery of active agents to biologically recognizable sites in vitro or in vivo requires a “biorecognition pair” consisting of a “biologically recognizable site”, usually a protein or a carbohydrate which is capable of reacting with a “biorecognition molecule”, usually a protein or a lectin, respectively, to form a unique complex. The wide range of events by which particular biologically recognizable sites uniquely complex with other molecules can include antibody-antigen binding reactions, hormone-receptor interactions, enzyme-substrate interactions, lectin/carbohydrate binding reactions and generally to ligand/receptor reactions. These interactions may also include complementary nucleic acid binding reactions such as DNA/DNA, RNA/DNA, RNA/RNA binding reactions, peptide nucleic acid/DNA binding reactions, PCR reactions, and DNA/protein reactions.

The term “biorecognition molecule” is used herein interchangeably with “targeting molecule” or “targeting moiety” and refers to the component of the biorecognition pair that recognizes and binds specifically to a biologically recognizable or target site. Thus, in the pair antigen-antibody, the biorecognition molecule is an antibody when the recognizable molecule is an antigen, and vice-versa; in the ligand-receptor pair, the biorecognition molecule is the ligand or the receptor; in the enzyme-substrate pair, the biorecognition molecule is the substrate or the enzyme, and the like.

According to the invention, the biorecognition or target molecule may be a peptide, a protein, a lipid, a carbohydrate, an oligonucleotide, a polynucleotide, or an organic molecule which binds to a target site.

In one embodiment, the biorecognition molecule is a peptide such as an oligopeptide containing 2-20 amino acid residues. The peptides can be natural or synthetic.

In another embodiment, the biorecognition molecule is a protein selected from, but not limited to, antibodies, antigens, hormones, cytokines, enzymes, receptors. Typical antibodies include monoclonal and polyclonal antibodies, fragments such as the Fab and Fc fragments, chimeric and humanized antibodies and derivatives thereof.

In another embodiment, the biorecognition molecule is a protein selected from, but not limited to, protamines, histones, albumins, globulins, phosphoproteins, mucoproteins, lipoproteins, nucleoproteins, and glycoproteins.

Examples of proteins for use in the present invention can include albumin, prealbumin, insulin, prolactin, antibodies to tumor cells or other disease states, alpha-1 lipoprotein, elastase inhibitors such as alpha-1 antitrypsin, transcortin, thyroxin-binding globulin, Gc-globulin, haptoglobin, erythropoietin, transferrin, hemopexin, plasminogen, immunoglobulin G, immunoglobulin M, immunoglobulin D, immunoglobulin E, immunoglobulin A, complement factors, oncoproteins, plasma proteins, rheumatoid factors prothrombin, parathyroid hormone, relaxin, glucagon, melanotropin, somatotropin, follicle stimulating hormone, luteinizing hormone, secretin, gastrin, oxytocin, vasopressin; enzymes such as cholinesterase, oxidoreductases, hydrolases, lyases and the like; interleukin such as IL-2; and growth factors such as EGF, TGF, and the like. Analogues and inhibitors derived from such materials are also encompassed by this invention.

Examples of lipids that can be used as biorecognition molecules are lipids with carbohydrate heads known as gangliosides. Other examples of biorecognition molecules are: haptens, biotin, biotin derivatives, lectins, galactosamine and fucosylamine moieties, receptors, substrates, coenzymes and cofactors; neuraminidases; viral antigens or hemagglutinins and nucleocapsids including those from any DNA and RNA viruses, bacterial antigens including those of gram-negative and gram-positive bacteria, fungal antigens, mycoplasma antigens, rickettsial antigens, protozoan antigens, parasite antigens, human antigens including those of blood cells, virus infected cells, genetic markers, heart diseases, cancer and tumor antigens such as alpha-fetoproteins, prostate specific antigen (PSA) and CEA, cancer markers and other oncoproteins. Other substances that can function as targeting moieties are certain proteins, hormones, vitamins such as folic acid, steroids, prostaglandins, synthetic or natural polypeptides, carbohydrates, antibiotics, drugs, digoxins, pesticides, narcotics, neurotransmitters, and substances used or modified such that they function as targeting moieties.

The active agent incorporated non-covalently into the cavity of the cyclodextrins and/or embedded/entrapped in the polymer matrix of the CD-containing polymer can be any type of molecule which will bring about a desired physical or chemical effect when incorporated in the cyclodextrin. This desired effect can be a label or reporter function which can be important when the bioactive protein locates and reacts with its bioactive mate or it can be a toxin or drug delivered specifically to a site of action by the biospecific reaction of the bound active agent and its biospecific mate. The biorecognition molecules facilitate delivery of the active agents to particular sites recognized by the biorecognition molecules Thus, the terms “active ingredient” or “active substance” or “active agent” are used herein interchangeably and refer to such a material that is either a label or marker or has biological activity that is therapeutic, inhibitory, antimetabolic, or preventive toward a disease such as cancer, an infectious disease (e.g., syphilis, gonorrhea, influenza) and heart disease or inhibitory or toxic toward any disease causing agent The active agent is located within the cavity of the cyclodextrin moiety and/or embedded within the CD-containing polymer matrix and may include one or more active agents and also non-active ingredients such as a plasticizer, and the like.

The active agent may be a drug including, but not limited to, prodrugs, anticancer drugs, antineoplastic drugs, antifungal drugs, antibacterial drugs, antiviral drugs, cardiac drugs, neurological drugs, and drugs of abuse. These drugs include alkaloids, antibiotics, bioactive peptides, steroids, steroid hormones, polypeptide hormones, interferons, interleukins, narcotics, nucleic acids, pesticides, prostaglandins, toxins and other materials known to have toxic properties to tissues or cells when delivered thereto including aflatoxins, ricins, bungarotoxins, illudins, chlorambucil, melphalan, 5-fluorouracil, procarbazine, lectins, irinotecan, ganciclovir, furosemide, indomethacin, chlorpromazine, methotrexate, cevine derivatives and analogs including cevadines, desatrines, veratridine, among others, and anticancer agents such as paclitaxel, cysplatin, doxorubicin and others.

The active agent can be a flavone derivative and analogs thereof including dihydroxyflavones, trihydroxyflavones, pentahydroxyflavones, hexahydroxyflavones, flavyliums, quercetins, fisetins.

The antibiotic active agent includes penicillin derivatives (i.e. ampicillin), tetracyclines, chlorotetracyclines, guamecyclines, macrolides (i.e. amphotericins, chlorothricin), anthracyclines (i.e. doxorubicin, daunorubicin, mitoxantrone), butoconazole, camptothecin, chalcomycin, chartreusin, chrysomicins (V and M), chloramphenicol, clomocyclines, cyclosporins, ellipticines, lilipins, fungichromins, griseofulvin, griseoviridin, methicillins, nystatins, chrymutasins, elsamicin, gilvocarin, ravidomycin, lankacidin-group antibiotics (i.e. lankamycin), mitomycin, and wortmannins

The active agent can be a purine or pyrimidine derivative and analogs thereof including 5′-fluorouracil 5′-fluoro-2′-deoxyuridine, and allopurinol; a photosensitizer including phthalocyanine, porphyrins and their derivatives and analogs; a steroid derivative and analogs thereof including estrogens, androgens, adrenocortical steroids, e.g., cortisones, estradiols, hydrocortisone, testosterones, prednisolones, progesterones, dexamethasones, beclomethasones and other methasone derivatives, cholesterols, digitoxins, digoxins and digoxigenins as well as steroid mimics such as diethylstilbestrol; a coumarin derivative and analogs including dihydroxycoumarins, dicumarols; chrysarobins, chrysophanic acids, emodins, secalonic acids; a dopa derivative and analogs including L-dopa, dopamine, epinephrine and norepinephrine; an alkaloid such as morphine, codeine and the like, ergot alkaloids, quinoline alkaloids and diterpene alkaloids; a barbiturate; amphetamines; and an anti-inflammatory agent such as prostaglandins, clofibric acid, indomethacin and the like.

Other specific active agents that can be used in accordance with the invention include drugs against infectious agents such as antiviral drugs against any DNA and RNA viruses, antibacterial drugs against both gram-negative and gram-positive bacteria, antifungal drugs, drugs against mycoplasma and rickettsia, antiprotozoan drugs, and antiparasitic drugs.

In another embodiment, the active agent is a label such as, but not limited to, radiolabeled compounds such as carbon-14- or tritium-labeled materials ranging from simple alkyls or aryls to more complicated species. Other labels can include azo dyes, enzyme and coenzyme labels, fluorescent labels such as fluoresceins, rhodamines, rosamines, rare earth chelates, and the like, chemiluminescent compounds such as luminol and luciferin, chemical catalysts capable of giving a chemical indication of their presence, electron transfer agents and the like.

In preferred embodiments of the invention, the targeting moiety is folic acid (vitamin B9) or a monoclonal antibody, particularly chimeric and humanized antibodies against cancers such as infliximab, basiliximab, abciximab, daclizumab, gemtuzumab, rituximab, trastuzumab, and others, and the active agent is an anticancer drug, such as doxorubicin or paclitaxel.

The biorecognition molecule/targeting moiety is linked covalently to the polymer backbone either directly or preferably via a spacer herein referred to also as a linking group. Preferred linking groups are polyether chains selected from polyethyleneglycol (PEG), preferably of MW 10-50,000 (PEG_{10-50,000}) or a polyetheramine such as poly(oxyethylene diamine O,O′-bis(2-aminopropyl)polypropylene glycol (e.g., the commercially available Jeffamine® D-230® or Jeffamine® D-400®, Huntsman) or O,O′-bis(2-aminopropyl)polypropylene glycol-block-polyethylene glycol-block-polypropylene glycol (e.g., Jeffamine® ED 600, Jeffamine® ED-900, Jeffamine® ED-2000), having the general formula H₂N—(CH(CH₃)—CH₂—O)ₓ—(CH₂CH₂—O)_{y}—(CH₂—CH(CH₃)—O)_{z}—CH₂CH(CH₃)—NH₂ (y may be ˜9 or 12.5 and (x+z) may be ˜3.6 or ˜6 for Jeffamine® ED-600, Jeffamine® ED-900, respectively).

In a more preferred embodiment, the linking group is PEG of MW 500-10,000 (PEG_{500-10,000}), most preferably PEG₃₃₅₀. In another more preferred embodiment, the linking group is Jeffamine® ED-900 or Jeffamine® ED-2000.

It is to be understood that according to the invention the active agent (“the guest molecule”) can be included within the cyclodextrin cavity and/or entrapped within the matrix of the CD-containing polymer used in the invention as the carrier molecule. Thus, small molecules will fit into the cavities provided by the cyclodextrins and may be located mainly there: smaller, less branched molecules will fit for inclusion in the alpha cyclodextrins, larger more branched materials for inclusion in the beta cyclodextrins and aromatics and other bulkier groups for inclusion within the gamma cyclodextrins. In all these cases, the active agent can be mainly located into the cavities of the CD residues but may also be entrapped within the matrix of the CD-containing polymer However, when the active agent is a large molecule such as a protein, e.g., an antibody, an antigen or an enzyme that do not fit into the cyclodextrin cavities, it will be entrapped within the polymer matrix of the CD-containing polymer and this is one of the advantages of the present invention with regard to the prior art described in U.S. Pat. No. 5,068,227.

Another advantage of the present invention relates to solubility issues. Many agents that are to be attached to biorecognition proteins are hydrophobic molecules and their attachment according to other technologies (not using cyclodextrins as carriers) decreases the solubility of the biorecognition molecule. Cyclodextrins confer increased solubility to the proteins and also help solubilize the complexed agent. Other hydroxyls on the cyclodextrins can be further derivatized to increase solubility if necessary

In one preferred embodiment, the polymer of the CD-containing polymer used in the conjugate of the present invention is a peptide or polypeptide wherein at least one of the amino acid residues of said peptide or polypeptide has a functional side group and at least one of the CD residues is covalently linked to said functional side group. Other CD residues may be linked to different functional side groups of other amino acid residues in said peptide or polypeptide chain and one or two CD residues may be covalently linked to the α-amino- and/or α-carboxy-terminal groups of said peptide or polypeptide. It should be understood that if only one CD moiety is attached to a peptide or polypeptide polymer, it is not linked to a terminal amino or carboxy group of said peptide or polypeptide. In some embodiments, all the amino acids of the peptide have side-chain functional groups and are bound through their side-chain functional groups to CDs and, thus, said peptide has no free functional side groups.

The peptide or polypeptide may be an all-L or all-D or an L,D-peptide or polypeptide, in which the amino acids may be natural amino acids, non-natural amino acids and/or chemically modified amino acids provided that at least one of such amino acids has a side-chain functional group. In a more preferred embodiment, the peptide or polypeptide comprises only natural amino acids selected from the 20 known natural amino acids that have a functional side group, namely, lysine, aspartic acid, glutamic acid, cysteine, serine, threonine, tyrosine and histidine.

The peptide or polypeptide may, according to another preferred embodiment, comprise one or more non-natural amino acids such as, but not limited to, an N_{α}-methyl amino acid, a C_{α}-methyl amino acid, a β-methyl amino acid, β-alanine (β-Ala), norvaline (Nva), norleucine (Nle), 4-aminobutyric acid (γ-Abu), 2-aminoisobutyric acid (Aib), ornithine (Orn), 6-aminohexanoic acid (ε-Ahx), hydroxyproline (Hyp), sarcosine, citruline, cysteic acid, statine, aminoadipic acid, homoserine, homocysteine, 2-aminoadipic acid, diaminopropionic (Dap) acid, hydroxylysine, homovaline, homoleucine, 1,2,3,4-tetrahydroisoquinoline-3-carboxylic acid (TIC), naphthylalanine (Nal), and a ring-methylated or halogenated derivative of Phe.

The peptide or polypeptide of the conjugate may further comprise chemically modified amino acids. Examples of said chemical modifications include: (a) N-acyl derivatives of the amino terminal or of another free amino group, wherein the acyl group may be a C₂-C₂₀ alkanoyl group such as acetyl, propionyl, butyryl, hexanoyl, octanoyl, lauryl, stearyl, or an aroyl group, e.g., benzoyl; (b) esters of the carboxyl terminal or of other free carboxyl groups, for example, C₁-C₂₀ alkyl, phenyl or benzyl esters, or esters of hydroxy group(s), for example, with C₂-C₂₀ alkanoic acids or benzoic acid; and (c) amides of the carboxyl terminal or of another free carboxyl group(s) formed with ammonia or with amines.

In one embodiment of the invention, the peptide is an oligopeptide of 2-20, preferably, 2-10, 2-5, 2-3, more preferably, 2 amino acid residues. The oligopeptide may be a homooligopeptide that is composed of identical amino acid residues. In preferred embodiments, the oligopeptide is a homodipeptide, more preferably Glu-Glu, Asp-Asp, Lys-Lys or Cys-Cys, and the conjugated CD-containing peptides are the polyglutamic acid peptides 24 and 26 and polyaspartic acid peptides 25 and 27 (Schemes 10 and 13, respectively) and the glutamic acid dipeptides 33 and 34 (Scheme 12).

In another embodiment, the polymer is a polypeptide or protein having 21 to 10,000, preferably, 100-1,000 or 100-500 amino acid residues. In a more preferred embodiment, the polypeptide is a homopolypeptide of an amino acid having a functional side group such as α- or ε-polylysine, α- or γ-polyglutamic acid, α- or β-polyaspartic acid, polycysteine, polyserine, polythreonine or polytyrosine. In one preferred embodiment, the polypeptide is polyaspartic acid. These polypeptides are commercially available.

According to another embodiments, the polypeptide of the conjugate of the invention is a synthetic random copolymer of different amino acids, wherein at least one of the amino acids has a functional side group, or it is a native, preferably inert, protein such as albumin, collagen, an enzyme such as a collagenase, a matrix metalloproteinase (MMPs) or a protein kinase such as Src, v-Src, a growth factor, or a protein fragment such as epidermal growth factor (EGF) fragment.

As used herein, the term “protein” refers to the complete biological molecule having a three-dimensional structure and biological activity, while the term “polypeptide” refers to any single linear chain of amino acids, usually regardless of length, and having no defined tertiary structure.

The CD-containing polymer used in the invention may also comprise a peptide or polypeptide covalently linked to a carbohydrate residue to form a glycopeptide, a glycopolypeptide or a glycoprotein. The carbohydrate residue may be derived from a monosaccharide such as D-glucose, D-fructose, D-galactose, D-mannose, D-xylose, D-ribose, and the like; a disaccharide such as sucrose and lactose; an oligo- or polysaccharide; or carbohydrate derivatives such as esters, ethers, aminated, aminated, sulfated or phospho-substituted carbohydrates. The glycopolypeptide may contain one or more carbohydrate residues. Some glycoproteins contain oligosaccharide residues comprising 2-10 monosaccharide units. The carbohydrate may be linked to a free amino group or carboxy group in the side chain of an amino acid residue, e.g., lysine, glutamic acid or aspartic acid via an N-glycosyl linkage, or to a free hydroxyl group of an amino acid residue, e.g., serine, threonine, hydroxylysine or hydroxyproline, via an O-glycosyl linkage. The glycopeptides and glycopolypeptides can be obtained by enzymatic or chemical cleavage of glycoproteins, or by chemical or enzymatic synthesis as well known in the art. Examples of glycoproteins useful according to the invention include collagens, fish antifreeze glycoproteins, lectins, hormones such as follicle stimulating hormone, luteinizing hormone, thyroid stimulating hormone, human chorionic gonadotropin, alpha-fetoprotein and erythropoietin (EPO), and proteoglycans (known also as glycosaminoglycans).

In another embodiment, the polymer consists of an oligonucleotide that may be a ribonucleotide or a deoxyribonucleotide oligonucleotide containing from 2 to 25 bases or the polymer is a ribonucleotide or a deoxyribonucleotide polynucleotide containing 26-1000 bases or more.

The CD in the conjugates of the invention may be a natural CD selected from α, β- and/or γ-CD and their combinations, analogs, isomers, and derivatives. The CD residues linked to the polymer may be identical or different. For example, the CD-containing polymer may comprise both α- and β-CD residues or any other combination of α-, β- and/or γ-CD residues. In preferred embodiments, the CD-containing polymer comprises only β-CD residues, and/or a β-CD derivative.

In one preferred embodiment, the cyclodextrin or cyclodextrin derivative is chemically modified prior to its bonding to an amino acid.

As used herein the terms “modified cyclodextrin” or “modified CD” or “CD derivative” are used interchangeably and refer to a cyclodextrin molecule which was chemically modified in order to facilitate its bonding to a side chain of an amino acid prior to polymerization, or to a functional side chain of an amino acid of the polymer backbone. This modification is carried out by replacing one or more hydroxyl groups) at position(s) 2, 3 and/or 6, preferably at position 6, of the CD molecule with a group selected from —NH₂, —NH(CH₂)ₘNH₂, —SH, —O(CH₂)ₘCOOH, —OC(O)(CH₂)ₘCOOH, —NH(CH₂)ₘCOOH, —NHC(O)(CH₂)ₘCOOH, —OC(O)(CH₂)ₘNH₂, —Br, —Cl, —I, or —OSO₂Ar, and Ar is a (C₆-C₁₄)aryl, preferably phenyl or tolyl and m is 1, 2, 3, 4 or 5.

Any cyclodextrin derivative which has at least one free hydroxyl group at position 6 or 2 or 3, preferably position 6 and can be modified as described above, is useful according to the invention. These derivatives include, but are not limited to, acetyl-CD; diacetyl-CD; carboxymethyl-CD; methylated or partially methylated —CD such as monomethyl-CD, dimethyl-CD, and cyclodextrins wherein only one of the hydroxyl groups in position 2 or 6 is not methylated; 2-hydroxyethyl-CD; 2-hydroxypropyl-CD; 2-hydroxyisobutyl-CD; β-CD sulfobutyl ether sodium salt; glucosyl-CD; and maltosyl-CD. Also preferred are oxidized cyclodextrins that provide aldehydes and any oxidized forms of any cyclodextrin derivatives that provide aldehydes or carboxylic acids.

Also included are higher homologues of cyclodextrins. For the purpose of this invention, individual cyclodextrin derivatives as well as molecules comprising two, three, four or multi cyclodextrin residues (herein sometimes referred to as dimer, trimer, tetramer or polymer, respectively) function as the primary structures for the synthesis of the cyclodextrin-containing polymer (peptide).

The CD derivatives are usually much more soluble than the native CDs. In addition, the derivatives formed by substitution with hydroxyalkyl groups have reduced toxicity and optimized solvent action.

For the preparation of the conjugates of the invention comprising a CD derivative as defined above, one should start with a modified CD derivative that is grafted onto the polymer or, alternatively, the derivatization of the CD residue may be carried out after grafting the modified CD onto a polymer.

In a more preferred embodiment, the native CD (α-, β- and/or γ-CD) or CD derivative is directly bonded to the amino acid through a free hydroxyl group, preferably at position 6, without first undergoing chemical modification. According to this embodiment, the cyclodextrin is bound directly, e.g., to the carboxyl functional side group of glutamic or aspatric acid via an ester bond. This amino acid-CD derivative is obtained by dire ct reaction between the CD and the diprotected amino acid, utilizing unique reaction conditions developed by the present inventors. These reaction conditions include the unique combination of EDC-HOBT-DMAP as coupling reagents and DMF as the solvent. According to this embodiment, the estaric bond to CD remains intact during deprotection of the α-amino and α-carboxyl groups provided that at least the N-protecting group is a benzylic moiety and catalitic hydrogeneation (H₂/C/Pd) is employed to remove the protecting groups.

It is well known that cyclodextrin hosts are capable of forming inclusion complexes by encapsulating guest molecules within their cavity, thus greatly modifying the physical and chemical properties of the guest molecule, mostly in terms of water solubility and chemical stability. Since the CDs are cyclic oligosaccharides containing 6-8 glucopyranoside units, they can be topologically represented as toroids (or doughnuts) wherein the larger and the smaller openings of the toroid (the secondary and primary hydroxyl groups, respectively) are exposed to the solvent. Because of this arrangement, the interior of the toxoids is not hydrophobic, but considerably less hydrophilic than the aqueous environment and thus is able to host hydrophobic molecules. On the other hand, the exterior is sufficiently hydrophilic to impart cyclodextrins (or their complexes) water solubility.

The CD-containing polymer of the conjugates of the invention is a system useful for the delivery of one or more kinds of active agents, for increasing the water solubility and improving the stability of water-insoluble active agents and/or as a mean for controlled release of the active agents. This system combines two categories of encapsulation: molecular encapsulation and microencapsulation. The CD residues attached to the polymer backbone serve as molecular encapsulators such that each CD residue (the host) forms an inclusion complex with a part of one molecule or with a whole molecule or with more than one molecule of the active agent (the guest). In addition, the polymer matrix as a whole can microencapsulate the active agent by embedding or entrapping molecules of the active agent within the matrix.

Thus, in accordance with the present invention, the active agent is either solely encapsulated within the cavity of the cyclodextrin residues (molecular encapsulation) or it is further, partially or completely, entrapped and/or embedded, i.e., microencapsulated, within the CD-containing polymer matrix.

The present invention, thus, further provides a method for combined micro- and molecular-encapsulation (nano-encapsulation) of an active agent in a sole carrier, said method comprises contacting (i.e., mixing, blending) said active agent with a conjugate of the invention, whereby the active agent is both encapsulated and entrapped within the cyclodextrin-containing polymer of said conjugate.

When the polymer is a peptide or polypeptide, controlled release of an active ingredient is triggered by the enzymatic degradation (enzymatic hydrolysis or dissociation) of the peptide or polypeptide, as they encounter specific enzymes at the target site. The hydrolyzing/digesting enzymes include all the proteases (proteinases, peptidases or proteolytic enzymes) that break peptide bonds between amino acids of proteins by proteolytic cleavage, a common mechanism of activation or inactivation of enzymes especially involved in blood coagulation or digestion. There are currently six classes of proteases: serine proteases, threonine proteases, cysteine proteases, aspartic acid proteases (e.g. plasmepsin), metalloproteases and glutamic acid proteases. The different proteases depend on the peptide or polypeptide sequence. Thus, chymotrypsin is responsible for cleaving peptide bonds following a bulky hydrophobic amino acid residue, preferably phenylalanine, tryptophan and tyrosine, which fit into a snug hydrophobic pocket. Trypsin comprises an aspartic acid residue at the base of a hydrophobic pocket and is responsible for cleaving peptide bonds following a positively-charged amino acid residue such as arginine and lysine on the substrate peptide to be cleaved. Elastase is responsible for cleaving peptide bonds following a small neutral amino acid residue, such as alanine, glycine and valine.

The dissociation of the peptide by the protease leads primarily to release of microencapsulated molecules, i.e. molecules embedded within the polymer matrix, and thus activates a first pulse or active ingredient release. This is followed by slow release, mainly of molecules encapsulated within the CDs. This advantageous two-phase release of active agents may be utilized to design and achieve unique effects in a wide variety of pharmaceutical applications. Thus, controlled release formulations may elicit release of active ingredients in two stages: (i) an initial pulse, releasing a substantial dose of the active ingredient, thus achieving an immediate effect; and (ii) continuous, controlled release, providing a prolonged effect of the active ingredient, over a, preferably predefined, number of hours.

The technology of the present invention can also be beneficial in targeted drug delivery of multiple types of drug molecules, to treat a variety of medical conditions. The unique structure and qualities of the encapsulation according to the invention offers the following unique benefits: (i) increased stability for large, unstable molecules such as insulin, allowing for a wider range of drug administration methods such as oral; (ii) delivery of water-insoluble active ingredients such as steroids; (iii) prevention of adverse effects by encapsulated delivery to the target site, for example, with anti-cancer chemotherapy drugs or antibiotics; (iv) highly specific targeting enabled by complexing the CD-containing polymers with additional ingredients, known to improve specificity and cell permeability such as hormones, antibodies or sugars; and (v) prevention of a contrast effect between drugs or other biologically active substances.

According to this embodiment, one or more kinds of active ingredients can be encapsulated and delivered simultaneously. Thus, for example, when the CD-containing polymer comprises two types of CD residues e.g., α- and β-CD, two kinds of active ingredients, which differ in molecular size, can be encapsulated within the same polymer. First, the larger molecules are contacted with the CD-containing polymer, resulting in occupation of the larger cavities of β-CD. Then, this CD-containing polymer is contacted with the smaller molecules, which are encapsulated by the smaller α-CD residues.

The present invention further provides the biorecognition molecule-CD-containing polymer compounds wherein the biorecognition molecules are covalently linked either directly or via a spacer to the end group of the polymer backbone. These compounds are useful as carriers for delivery of active agents/drugs to the target sites recognized by the biorecognition molecules.

The present invention further provides pharmaceutical compositions comprising the conjugates of the invention.

The conjugates are obtained by mixing the active agent with the delivery system consisting of the CD-containing polymer and the biorecognition molecule. The obtained liquid solution may be mixed with pharmaceutically acceptable excipients or diluents or it may be first dried and then mixed with pharmaceutically acceptable excipients or diluents and then formulated as pharmaceutical composition in any suitable form for administration, for example, as liquid preparations for oral or parenteral administration or as solid preparations, e.g., tablets, capsules, etc.

The invention further provides a method for delivering an active agent to a target site recognized by a biorecognition molecule, which comprises administering to an individual in need a conjugate of the invention.

The present invention provides, in another aspect, processes for producing the conjugates of the invention. The synthesis of the starting compounds CD-amino acid derivatives and CD-containing peptides and polypeptides is fully described in the above-mentioned WO 2007/072481 of the same applicant.

One process comprises a first step of modification of the CD prior to its binding to a functional side group of an amino acid, as depicted schematically in Schemes 1-3 herein. The preparation of a modified CD is carried out by replacement of one or more hydroxyl groups (—OH) at positions 2, 3 and/or 6 with one or more functional groups Z selected from —NH₂, —NH(CH₂)ₘNH₂, —SH, —O(CH₂)ₘCOOH, —OC(O)(CH₂)ₘCOOH, —NH(CH₂)ₘCOOH, —NHC(O)(CH₂)ₘCOOH, —OC(O)(CH₂)ₘNH₂, halogen such as Cl, Br or I, or —OSO₂Ar, wherein Ar is a (C6-C10) aryl, preferably phenyl or tolyl, and m is 1, 2, 3, 4 or 5, as depicted in Scheme 1.

An example of a such modified β-CD compound is mono-6-deoxy-6-amino-β-CD, herein designated compound 4, wherein the 6-hydroxyl group is replaced with an amino group to obtain the compound as depicted in Scheme 2.

Another example of a modified β-CD is the compound mono-6-deoxy-6-(2-aminoethyl)amino-β-CD, herein designated compound 5, wherein the hydroxyl of β-CD is replaced with ethylenediamino group as depicted in Scheme 3.

In another preferred process, the conjugates of the invention are prepared starting with an unmodified α-, β- or γ-CD, herein termed “native CD”, which is directly linked to a free carboxy group of a functional side chain of a diprotected amino acid through its OH group at position 6, or 3 or 2.

When the backbone polymer is a peptide or a polypeptide, the CD-containing polymer can be prepared using one of the three alternative methods below:

(i) covalently linking a native CD or modified CD to the free functional side group of a diprotected amino acid residue X—CH—(COOR₁)(NHR₂), wherein R₁ and R₂ are carboxyl and amino protecting groups, respectively, and the amino acid may be aspartic acid, glutamic acid, serine, tyrosine, lysine, cysteine, and the like, to produce the CD-amino acid derivative, as depicted in Scheme 4. Then, deprotection is carried out and the obtained derivative is polymerized to give the corresponding CD-containing peptide or polypeptide, as shown in Scheme 5;

(ii) covalently grafting a native CD or a modified CD directly to one or more functional side groups of amino acids of a desired peptide, polypeptide or protein chain, as shown in Scheme 6. For a polypeptide of 5-1000 amino acids, this process may result in 50-70% of random CD binding to the peptide backbone; or

(iii) coupling a free α-amino group of a CD-amino acid derivative with a free α-carboxy group of a second CD-amino acid derivative to give the corresponding CD-containing dipeptide as shown in Scheme 7. This method is suitable for the preparation of CD-containing oligopeptides of up to 10 amino acid residues, preferably 4, more preferably 2 amino acid residues, wherein each of the amino acids in the oligopeptide is covalently bound to a CD residue through its functional side group.

Diprotection of amino acids can be effected by blocking the α-amino and α-carboxy groups using approaches known in the art. Thus, the amino group may be blocked by tert-butyloxycarbonyl(t-Boc) or benzyloxycarbonyl protecting group, and the free carboxy group may be converted to an ester group e.g., methyl, ethyl, tert-butyl or benzyl ester.

Deprotection of the α-amino and α-carboxy groups is usually carried out under conditions that depend on the nature of the protecting groups used. Thus, benzyloxycarbonyl and benzyl groups are displaced by hydrogenation in the presence of Pd/C, and t-Boc groups are cleaved in the presence of trifluoroacetic acid or HBr/CH₃COOH at room temperature. The methyl, ethyl, tert-butyl or benzyl ester groups may be removed by saponification in the presence of sodium hydroxide (NaOH) or potassium hydroxide (KOH) solution or concentrated ammonium hydroxide (NH₄OH) solution.

It was discovered by the present inventors that deprotection of a CD-amino acid derivative (CD-AA), wherein the CD is directly bound via an ester bond to a diprotected amino acid, may not destroy this ester bond provided that both the amino- and carboxy-protecting groups comprise a benzyl moiety, and the deprotection is carried out under catalytic hydrogenation (H₂/C/Pd in methanol/water).

Polymerization of the amino acids can be performed according to any suitable process known in the art for peptide polymerization. Prior to polymerization, either the α-amino or the α-carboxy group is protected, thus controlling the direction of peptide bond formation and the nature of the polymer synthesized. Homo- and hetero-polymers can be obtained using the same polymerization process. The resulting polymer's identity and length are determined by the kind and amount of amino acids introduced into the reaction batch and depend on the polymerization reaction conditions such as the amount of coupling agent, concentration of the reactants, reaction temperature and stirring rate.

When different amino acids are employed in the polymerization process, a mixture of different peptides is obtained. These peptides differ in constitution and size. In the polymerization of homopeptides, peptides of different sizes are obtained. The peptides are separated based on their molecular size or weight using filtration means well known in industrial polymerization processes. For example, fractional isolation and purification of the peptides mixture may be carried out using a suitable membrane (dialysis tube) such that peptides having a given range of molecular weights are isolated depending on the pore size of the membrane.

After the cyclodextrin-containing polymer is synthesized, it is coupled to the desired targeting moiety. In one preferred embodiment, the targeting moiety is linked directly to the CD-containing polymer. According to a more preferred embodiment, the targeting moiety is activated first by binding at least one functional group selected from —COOH, —NH₂, —SH, or —OH of said moiety with a leaving group.

In a more preferred embodiment, the targeting moiety is linked to the CD-polymer through a spacer or a linking group as defined above. The linking group and targeting moiety may be combined together first, and then conjugated covalently to the CD-polymer. Alternatively, the CD-polymer may first be combined with the linking group followed by its conjugation via the linking group to the targeting moiety.

The coupling of the two components as defined above may be carried out by three alternative synthesis approaches. According to the first approach, the targeting moiety is first activated by binding at least one functional group selected from —COOH, —NH₂, —SH, or —OH of said moiety with a leaving group and then contacting the activated targeting moiety with the linking group. The linking group-targeting moiety product is then reacted with a CD-amino acid (AA) or with a CD-peptide under reaction conditions that allow linking of the targeting moiety to at least one free functional group (—COOH, —COO⁻, —NH₂ or —SH group) of the peptide or polypeptide, to produce the desired targeting moiety-linking group-CD-containing polymer compound.

According to the second approach, the targeting moiety is linked directly to the linking group in a process which does not involve prior activation of the targeting moiety and the resulting targeting moiety-linking group compound is reacted with the CD-containing polymer as described above.

According to the third approach, the CD-AA or CD-peptide is interacted directly with an excess amount of the linking group, and the resulting product is reacted with the activated or non-activated targeting moiety to obtain the final product wherein the targeting moiety is linked to at least one free functional group (—COOH, —COO⁻, —NH₂ or —SH) of said amino acid derivative or peptide or polypeptide.

In preferred embodiments of the present invention, the targeting moiety is folic acid (FA) and the linking group is a polyether, preferably PEG, or a polyether amine such as a Jeffamine.

In one preferred embodiment, folic acid (FA) is first activated by esterification, with the leaving group NHS in the presence or DMSO and DCC to obtain the intermediate FA-NHS. In a more preferred embodiment, the activated FA is reacted directly with a CD-AA or a CD-peptide, e.g., polyGlu or polyAsp.

In another more preferred embodiment, the activated FA is reacted with excess PEG or Jeffamine of different molecular weights (i.e., different lengths) to obtain the conjugate PEG-FA or Jeffamine-FA. This product is then further conjugated with an amino acid-CD derivative (CD-AA) or with CD-peptide in DMSO in the presence of EDC HOBT and DMAP to obtain the final product, the conjugate CD-AA/peptide-PEG-FA or CD-AA/peptide-Jeffamine-FA. The yield using this synthetic approach is not high.

In another preferred embodiment, FA is interacted directly with excess PEG or Jeffamine of different lengths in the presence of DMSO and PyBOP (with or without) HOBT and DMAP) to obtain the conjugate PEG-FA or Jeffamine-FA, respectively. This product is then further conjugated with CD-AA or with CD-peptide to obtain the final product, the conjugate CD-AA/peptide-PEG-FA or CD-AA/peptide-Jeffamine-FA.

In a most preferred embodiment, the CD-AA or CD-peptide is interacted directly with excess PEG or Jeffamine of different molecular weights in the presence of DMSO and PyBOP (with or without HOBT and DMAP) to obtain the conjugate CD-AA-PEG or CD-peptide-Jeffamine, respectively. This product is then further conjugated with folic acid to obtain the final product, the conjugate CD-AA/peptide-PEG-FA or CD-AA/peptide-Jeffamine-FA. Purification of the product is carried out by dialysis in order to remove traces of folic acid. The yield using, this synthetic approach is the highest

In one preferred embodiment, a native CD (i.e., an unmodified CD) such as α-CD, β-CD or γ-CD, is covalently linked to a free functional carboxy group of a diprotected amino acid to form a CD-diprotected amino acid derivative wherein the CD is directly linked to said carboxy group via an ester bond.

In another more preferred embodiment, the method (i) is used for the production of conjugates comprising CD-containing homopeptides. More preferably, the peptide is an oligopeptide comprised of glutamic acid-CD or aspartic acid-CD or lysine-CD monomers such as the herein designated homo-oligopeptides 24-27 (Scheme 10).

In another preferred embodiment, a CD-containing peptide, polypeptide or protein is produced according to method (ii) above by covalently grafting a native CD or a modified CD directly to one or more functional side groups of amino acids of a desired peptide, polypeptide or protein. In a more preferred embodiment, the method (ii) is used for alografting mono-amino- and ethylenediamino-CD and ethylcarboxy-CD derivatives to polyglutamic acid (poly-Glu) or polyaspartic acid (poly-Asp) or polylysine (poly-Lys) to obtain CD-containing polypeptides. One such preferred polypeptide is the poly-Asp polypeptide herein designated 37 (Scheme 15), in which 50% of the carboxyl groups are grafted with mono-amino-CDs. In a most preferred embodiment, the conjugate which comprises 37 is the conjugate depicted in Scheme 16, herein designated conjugate 38, in which said poly-Asp-CD polypeptide is linked via PEG to folic acid.

The di-coupling method mentioned above may be carried out with native CDs such as α-CD, β-CD or γ-CD, and the CD is linked to the carboxy side group of the diprotected amino acid via an ester bond. In that case, both N- and carboxy-protecting groups comprise a benzyl moiety.

The di-coupling method is preferably used for the production of conjugates comprising CD-dipeptides, more preferably CD-homo-dipeptides, most preferably the Glu(monoamino β-CD)-Glu(mono amino β-CD) derivatives, herein identified as dipeptides 33 and 34.

The conjugate of the invention comprising an active agent encapsulated within the CD residue and/or embedded within the polymer matrix is prepared by mixing the active agent with the CD-containing polymer conjugated to a targeting moiety either directly or via a linking group, acting as a carrier. The carrier may be prepared beforehand and stored at room temperature or at a lower temperature. The mixing can be carried out by completely dissolving both components in water or in a mixture of ethanol/methanol and water and stirring at room temperature for up to three days. The ethanol/methanol is then evaporated and uncomplexed active agent is removed by filtration.

The present invention further provides a tri-CD-dipeptide, wherein two amino acid are linked to three cyclodextrin residues, such that two of the CD are linked to the two functional side chains and the third CD is linked to the α-carboxy or α-amino group. The dipeptide may be prepared either according to method (i) or by the di-coupling method (iii) mentioned above. In one preferred embodiment, the tri-CD-dipeptide is (β-CD)-Glu(β-CD)-Glu(β-CD) derivative Glu depicted in Scheme 14 and designated herein 36, wherein the β-CD is mono amino β-CD.

Further provided by the present invention are conjugates comprising a targeting moiety and a tri-CD-dipeptide containing an active agent encapsulated within the cavities of the cyclodextrin residues and within the cavity or pouch formed by the amino acid and the two CD residues. The tri-CD-dipeptide is prepared from a di-CD-AA, and a CD-AA derivative, which in turn may be preferred according to any one of methods (i)-(iii) above. In a more preferred embodiment, the di-CD-Glu herein designated 31 is reacted with CD-glutamic acid, herein designated 16, as depicted in Scheme 12. The active agent may be a drug.

For preparation of the carrier function, i.e., tri-CD-di-AA-linker-targeting moiety, the tri-CD-di-AA is first activated and then linked to the targeting moiety via a linking group. In to a more preferred embodiment, (β-CD)-Glu(β-CD)-Glu(β-CD) is reacted with the activating agent succinic anhydride such that the succinic ring is opened and is bound at one end through an amide bond to a free amino group of the dipeptide and the other end in a carboxylic group free to react with the linking group and then with targeting moiety. In one preferred embodiment, the linking group is Jeffamine ED 900 and the targeting moiety is FA. The active agent may be doxorubicin or paclitaxel.

It was previously discovered by the present inventors, as mentioned in WO 2007/072481, that covalent linking of two or three residues of cyclodextrin to one molecule of amino acid selected from aspartic acid, glutamic acid and lysine, produce a compound with a further ‘pouch’ for encapsulation of active agents. Since these compounds have no peptidic bond, they are not affected by protease degradation in the body and can thus form very stable complexes with active agents. Such compositions will cross the stomach and the small intestine without degradation.

Thus, a further aspect contemplated by the present invention are conjugates comprising an active agent and derivatives comprising two residues of a CD covalently linked to one molecule of amino acid, herein identified as “di-CD-amino acid derivative”, which in turn is linked either directly or via a linking group to a targeting moiety. The amino acid may be glutamic acid, aspartic acid or lysine.

The process for production of such di-CD-amino acid derivatives is described in WO 2007/072481 and depicted in Scheme 11. In one embodiment, two modified CDs, e.g. compound 4 are reacted with a N-protected amino acid, e.g., the protected glutamic acid 29, thus obtaining the N-protected di-CD-amino acid derivative herein designated 28, and deprotection leads to the di-CD-amino acid derivative designated herein 31. In another embodiment, the two modified CDs 5 are reacted with the N-protected glutamic acid 29, thus obtaining the N-protected di-CD-amino acid derivative designated 30, and deprotection leads to the di-CD-amino acid derivative 32.

In one preferred embodiment, the di-CD-amino acid derivative is 31, which is activated by linking succinic anhydride to a free amino group, followed by linking the succinic derivative to Jeffamine ED 900 and then to FA. The active agent hosted within the cavity or pouch formed by the amino acid and the two CD residues is, for example, doxorubicin or paclitaxel.

The conjugates comprising the di-CD-amino acid and tri-CD-amino acid derivatives with the encapsulated ingredient may be used for all applications as described hereinbefore for conjugates comprising CD-containing peptides and polypeptides.

In preferred embodiments of methods (i) and (iii), in step (ii), the amino acid-CD derivative is obtained by reacting an α-amino acid selected from glutamic aspartic acid, lysine or cysteine, most preferably glutamic or aspartic acid or lysine, in the L, D or racemic form with a native or modified CD in water or an organic solvent such as dimethylformamide (DMF) or dimethylsulfoxide (DMSO) or a mixture of water, DMF and DMSO in the presence of an excess of a dehydrating agent such as dicyclohexylcarbodiimide (DCC), N-β-dimethylaminopropyl)-N′-ethyl-carbochiimide hydrochloride (EDC), (benzotriazol-1-yloxy)tripyrrolidino phosphonium hexafluoro phosphate (PyBOP) and a catalyst such as 1-hydroxybenzotriazole (HOBT), pyridine, 4-dimethylaminopyridine (DMAP), triethylamine. Diisopropylethylamine (DIPEA), clay or zeolite. The reaction is generally carried out with stirring at a temperature between 0° C. to 50° C. until the starting materials have completely disappeared and the mixture is then filtered. Following concentration under vacuum, the amino acid-CD derivative is recrystallized, preferably from water or water-ethanol or water-methanol.

Amino acid-CD derivatives, prepared according to the methods described above from modified or non-modified CDs are intermediates in the processes for the preparation of the conjugates of the invention. The amino acid-CD derivative may be mono(6-aminoethylamino-6-deoxy)cyclodextrin covalently linked via the 6-position CD—NH—CH₂—CH₂—NH-group to the functional side group of an α-amino acid selected from aspartic acid, glutamic acid, lysine, tyrosine, cysteine, serine, threonine and histidine. Examples of such derivatives are represented by the compounds herein identified as 10, 11, 14, 15, 18 and 19.

The amino acid-CD derivative may also be a mono(6-amino-6 deoxy)cyclodextrin covalently linked via the 6-position CD-NH— group to the functional side group of an α-amino acid selected from aspartic acid, glutamic acid, lysine, tyrosine, cysteine, serine, threonine and histidine, wherein the α-amino or both the α-amino and the α-carboxy groups are protected. Examples of such derivatives are represented by the compounds herein identified as 6, 8, 16, and 17.

Schemes 8-10 herein, depict the amino acid-CD derivatives mentioned above, namely: the diprotected glutamic acid-CD derivatives 6, 10; the diprotected aspartic acid-CD derivatives 8, 11; the α-carboxy protected glutamic acid-CD and aspartic acid-CD derivatives 14 and 15, respectively; the α-amino protected glutamic acid-CD derivatives 16, 18; the α-amino protected aspartic acid-CD derivatives 17, 19; and the glutamic acid-CD and aspartic acid-CD derivatives 22 and 23, respectively.

The invention will now be illustrated by the following non-limiting Examples.

### EXAMPLES

In the Examples herein, conjugates and intermediates will be presented by their respective Arabic numbers in bold according to the following List of Compounds. CD-amino acid derivatives and CD-polypeptides 1-35 are described in WO 2007/072481 and their synthesis is fully disclosed therein. For some of these compounds, the synthesis is described herein in the examples. Schemes 1-13 depict the synthesis of compounds disclosed in WO 2007/072481, and Schemes 14-16 describe the synthesis of the CD-amino acid derivative 36, CD-polymer 37 and conjugate 38, respectively. The schemes are presented at the end of the description, just before the References.

### List of Compounds

1. β-cyclodextrin (β-CD or CD)
2. Mono-6-deoxy-6-(p-toluenesulfonyl)-β-cyclodextrin (mono-tosyl-CD)
3. Mono-6-deoxy-6-azido-β-cyclodextrin (mono-azido-CD)
4. Mono-6-deoxy-6-amino-β-cyclodextrin (mono-amino-CD)
5. Mono-6-deoxy-6-(2-aminoethylamino)-β-cyclodextrin (mono-ethyldiamino-CD)
6. Mono-6-deoxy-6-[4-(benzyloxycarbonyl)-4-(tert-butyloxycarbonylamino) butyrylamino]-β-cyclodextrin
7. 4-(benzyloxycarbonyl)-4-(tert-butyloxycarbonylamino) butyric acid (N-Boc-L-glutamic acid-1-benzyl ester)
8. Mono-6-deoxy-6-[3-(benzyloxycarbonyl)-3-(tert-butyloxycarbonylamino) propionylamino]-β-cyclodextrin
9. 3-(benzyloxycarbonyl)-3-(tert-butyloxycarbonylamino)propanoic acid (N-Boc-L-aspartic acid-1-benzyl ester)
10. Mono-6-deoxy-6-[4-(benzyloxycarbonyl)-4-(tert-butyloxycarbonylamino) (butyroylamino ethane)amino]-β-cyclodextrin
11. Mono-6-deoxy-6-[3-(benzyloxycarbonyl)-3-(tert-butyloxycarbonylamino) (propionylamino ethane)amino]-β-cyclodextrin
12. Mono-6-deoxy-6-[4-(benzyloxycarbonyl)-4-amino butyryl amino]-β-cyclodextrin
13. Mono-6-deoxy-6-[3-(benzyloxycarbonyl)-3-amino propionyl amino]-β-cyclodextrin
14. Mono-6-deoxy-6-[4-(benzyloxycarbonyl)-4-amino(butyrylamino ethane)amino]-β-cyclodextrin
15. Mono-6-deoxy-6-[3-(benzyloxycarbonyl)-3-amino(propionylamino ethane)amino]-β-cyclodextrin
16. Mono-6-deoxy-6-[4-carboxy-4-(tert-butyloxycarbonylamino)butyrylamino]-β-cyclodextrin
17. Mono-6-deoxy-6-[3-carboxy-3-(tert-butyloxycarbonylamino)propionylamino]-β-cyclodextrin
18. Mono-6-deoxy-6-[4-carboxy-4-(tert-butyloxycarbonylamino)(butyrylamino ethane)amino]-β-cyclodextrin
19. Mono-6-deoxy-6-[3-carboxy-3-(tert-butyloxycarbonylamino)(propionylamino ethane)amino]-β-cyclodextrin
20. Mono-6-deoxy-6-[4-carboxy-4-amino butyrylamino]-β-cyclodextrin ((mono amino β-CD)-Glu)
21. Mono-6-deoxy-6-[3-carboxy-3-amino propionylamino]-β-cyclodextrin
22. Mono-6-deoxy-6-[4-carboxy-4-amino(butyrylamino ethane)amino]-β-cyclodextrin
23. Mono-6-deoxy-6-[3-carboxy-3-amino(propionylamino ethane)amino]-β-cyclodextrin
24. poly[mono-6-deoxy-6-[4-carboxy-4-amino butyrylamino]β-cyclodextrin]
25. poly[mono-6-deoxy-6-[3-carboxy-3-amino propionylamino]-β-cyclodextrin]
26. poly[mono-6-deoxy-6-[4-carboxy-4-amino(butyrylamino ethane)amino]-β-cyclodextrin]
27. poly[mono-6-deoxy-6-[3-carboxy-3-amino(propionylamino ethane)amino]-β-cyclodextrin]
28. 2-(tert-butyloxycarbonylamino)-N¹,N⁵-bis(6-mono-6-deoxy-β-cyclodextrin) pentanediamide
29. 4-carboxy-4-((tert-butyloxy)carbonyl)aminobutyric acid (N-Boc-L-glutamic acid)
30. 3-(tert-butyloxycarbonylamino)-N¹,N⁶-bis(2-((6-mono-6-deoxy-β-cyclodextrin)amino)ethyl)-2-oxohexanediamide
31. 2-amino-N¹,N⁵-di(6-mono-6-deoxy-β-cyclodextrin) pentanediamide
32. 3-amino-N¹,N⁶-bis(2-((6-mono-6-deoxy-β-cyclodextrin)amino)ethyl)-2-oxohexanediamide
33. Glu(mono amino β-CD)-Glu-(mono amino β-CD) (See Scheme 12).
34. (Mono amino β-CD)-Glu-Glu

### 35. CD-polyAsp

36. Tri-(mono amino β-CD)-Glu-Glu
37. (Mono amino β-CD)₅₀-polyGlu (See Scheme 15)
38. [(mono amino β-CD)-poly-Glu]-PEG₃₃₅₀-Folic acid
39. (mono amino β-CD)-Glu-Jeffamine
40. (Mono amino β-CD)-Glu-Jeffamine-folic acid
41. Di-(mono amino β-CD)-Glu-SA
42. Di-(mono amino β-CD)-Glu-Jeffamine 42
43. Di-(mono amino β-CD)-Glu-SA-Jeffamine-Folic acid
44. (mono amino β-CD)₂-Glu-Glu-Jeffamine
45. (Mono amino β-CD)₂-Glu-Glu-Jeffamine-Folic acid
46. Tri-(mono amino β-CD)-Glu-Glu-SA
47. Tri-(mono amino β-CD)-Glu-Glu-SA-Jeffamine
48. Synthesis of tri-(mono amino β-CD)-Glu-Glu-SA-Jeffamine-FA

### 49. CD-polyAsp-Jeffamine

50. CD-polyAsp-Jeffamine-Folic acid
51. Mono-6-deoxy-6-(4-carboxy-4-amino butyrate)-β-cyclodextrin
52. Mono-6-deoxy-6-(3-carboxy-3-amino propionate)-β-cyclodextrin
53. Mono-6-deoxy-6-(butyroylamino ethoxy)-β-cyclodextrin
54. Mono-6-deoxy-6-(propionylamino ethoxy]-β-cyclodextrin

### 55. Di-CD-Glu-PEG₃₃₅₀-FA-RhB

### 56. Tri-CD-Glu-Glu-PEG₃₃₅₀-FA-RhB

### 57. CD-polyGlu-PEG₃₃₅₀-FA-RhB

### Materials and Methods

Chemicals. Cyclodextrins (Aldrich) were dried (12 h) at 110° C./0.1 mmHg in the presence of P₂O₅. Amino acid derivatives were obtained from Aldrich, Sigma or Fluka and were used without further purification. Acetone (CH₃COCH₃, HPLC-grade, Tedia), acetonitrile (CH₃CN, HPLC-grade, Tedia), methanol (CH₃OH, HPLC-grade, Tedia), water (H₂O, HPLC-grade, Tedia), dimethylformamide (DMF, anhydrous, 99.8%, Aldrich), dimethyl sulfoxide (DMSO, 99.9%, Aldrich), n-butanol (n-BuOH, 99%, Fluka), iso-butanol (iso-BuOH, 99%, Riedel-deHaen), n-hexane (99.5%, Frutarom), diethyl ether (99.5%, Frutarom), ethyl acetate (EtOAc, 99.5%, Frutarom), dichloromethane (DCM, 99.5%, Frutarom), ammonium hydroxide (NH₄OH, 25% NH₃, Frutarom), p-Toluenesulfonylchloride (TsCl, 99+%, Aldrich), 4,4-Dimethyl aminopyridine (DMAP, 99%, Aldrich), N,N-dicyclohexylcarbodiimide (DCC, 99%, Fluka), N-(3-dimethylaminopropyl)-N′-ethyl-carbodiimide hydrochloride (EDC, 98%, Fluka), (benzotriazol-1-yloxy)tripyrrolidino phosphonium hexafluoro phosphate (PyBOP, 97%, Fluka), 1-Hydroxybenzotriazole (HOBT, Aldrich), succinic anydride (99%, Aldrich), potassium iodide (KI, Yavin-Yeda), sodium hydroxide (NaOH, 99%. Merck) and magnesium sulphate (MgSO₄, anhydrous, 98-100%. Bio-Lab) were used without further purification. Zeolites were dried at 400° C. under atmospheric pressure for 4 h. Column chromatography was performed using silica gel 60 (0.040-0.063 mm) (Merck) or LiChroprep RP-18 (40-63 μm, Merck) for column chromatography. TLC analysis were performed on silica gel 60 TLC plates and silica gel 60 F₂₅₄ PLC plates (Merck) with EtOAc:2-propanol:NH₄OH_{(aq)}:water (7:7:5:4) or 1-butanol: ethanol:NH₄OH_{(aq)}:H₂O (4:5:6:3) or 1-butanol:ethanol:NH₄OH_{(aq)}(4:5:6) eluents. Cyclodextrin derivatives were detected by spraying with 5% v/v concentrated sulfuric acid in ethanol and heating at 150° C. or iosine (I₂). ¹H-NMR and ¹³C-NMR spectra were recorded on an FT-200 MHz spectrophotometer with deuterated dimethyl sulfoxide (DMSO) or deuterated water (D₂O) or deuterated chloroform (CDCl₃) as a solvent; chemical shills were expressed as δ units (ppm). HPLC analysis were performed on Thermo instrument equipped with UV- and LSD-detector. The column used was a Luna 5 u NH₂ column (100A, size 250-4.6 mm), mobile phase: acetonitrile/H₂O, and flow 1.2 ml/min.

Cell culture. KB cells (ATCC CCL-17) were obtained from ATCC and grown on Minimum essential medium (Eagle) with 2 mM L-glutamine; 0.1 mM non-essential amino acids; 0.2 Earle's BSS adjusted to contain 1.5 g/l sodium bicarbonate; and 1.0 mM sodium pyruvate, 90%; heat inactivated fetal bovine serum, 10%. Cells were subcultured according to the ATCC recommended protocol. After 3 cycles of splitting at 85% confluence, 2,000 to 50,000 cells were seeded on transparent 96 well plate. Following 24 hours it was decided that optimal conditions would be seeding 35,000 cells per well for assay to be carried out in the following day.

### Example 1

### Synthesis of compound 40 (mono amino β-CD)-Glu-Jeffamine-folic acid

The title compound was prepared starting from deprotection of compound 6, which, in turn, was synthesized as described in WO 2007/072481

### i. Synthesis of compound 20

The compound 20 (mono-6-deoxy-6-[4-carboxy-4-amino butyrylamino]-β-cyclodextrin) also termed herein (mono amino(1-CD)-Glu was obtained by removing the N-protecting Boc group and benzyl group from compound 6 as shown in Scheme 10, as follows:

Compound 6 (1.453 g, 1.0 mmol) was dissolved in TFA (5 ml) and CH₂Cl₂ (5 ml) and the mixture was stirred at 25° C. for 3 h. The solvent was removed by evaporation under reduced pressure (<25° C.). The residue was dissolved in 1M NaOH (20 ml) and the mixture was stirred at 25° C. for 5 h. The solvent was removed by evaporation under reduced pressure (<25° C.) and the residue was poured into methanol (200 ml). The white precipitate was filtered and dried under vacuum (65% yield). TLC analysis of 20 performed on silica plates (EtOAc:2-propanol:conc. NH₄OH:water-7:7:5:4) showed one major spot (R_{f}=0.20). ¹H NMR (D₂O) δ: 1.8-2.2 (m, 4H), 3.47-3.84 (m, 42H), 4.9-5.1 (m, 7H).

### ii. Synthesis of (mono amino β-CD)-Glu-Jeffamine 39

O,O′-bis(2-aminopropyl)-polypropylene-glycol-block-polyethylene-glycol-block-polypropylene-glycol (Jeffamine® ED-900) (2.70 gr, 3.0 mmol) and 20 (1.0 mmol) were dissolved in DMF (10 ml), followed by the addition of PyBOP (0.52 gr, 1.0 mmol). The reaction mixture was stirred at room temperature for 2 h, then another portion of PyBOP (0.52 gr., 1.0 mmol) was added and the stirring was continued overnight. DMF was removed by rotary evaporation. Methanol (5 ml) was added to the reaction mixture and the resulting solution was poured into ethyl acetate (100 ml). The white precipitate was filtered and dried under reduced pressure (1.61 gr, 74% yield).

### iii. Synthesis of (mono amino β-CD)-Glu-SA 40

The (mono amino β-CD)-Glu-Jeffamine (1.0 mmol) obtained above and folic acid (FA, 0.882 gr., 2.0 mmol) were dissolved in anhydrous DMSO (20 ml). PyBOP (0.52 gr, 1.0 mmol) was added and the reaction mixture was stirred at room temperature for 2 h, then another portion of PyBOP (0.52 gr., 1.0 mmol) was added and the stirring was continued overnight. The reaction mixture was poured into diethyl ether (250 ml). The oily orange precipitate was separated from the solution, dissolved in water (10 ml) and centrifuged to remove trace insolubles. The supernatant was dialyzed in Spectra/Por CE tubing (MW cutoff 1000) against distilled water (3×1000 ml). The dyalizate is lyophilized and the residue dried in vacuo over P₂O₅. The yield is 81%.

### Example 2

### Synthesis of di-(mono amino β-CD)-Glu-SA-Jeffamine-folic acid derivative 43

The title derivative was synthesized starting from di-(mono amino β-CD)-Glu derivative 28, which was obtained by coupling one molecule of N-protected glutamic acid 29 (N-Boc-L-glutamic acid) with two moieties of compound 4 (mono-6-deoxy-6-amino-β-cyclodextrin), using DCC and HOBT in DMF (mono amino-CD:amino acid 2:1). 28 was then deprotected by removing the N-protecting Boc group using TFA in CH₂Cl₂ the preparation of 28 and 31 is described in WO 2007/072481 and shown in Scheme 11 herein.

### 1. Synthesis of di-(mono amino β-CD)-Glu-SA 41

di-CD-Glu 31 (1.0 mmol) and DMAP (0.12 gr, 1.0 mmol) were dissolved in DMF (5 ml). Succinic anydride (0.10 gr, 1.0 mmol) was added and the reaction mixture was stirred at 25° C. for 5 h.

### ii. Synthesis of di-(mono amino β-CD)-Glu-SA-Jeffamine 42

O,O′-bis(2-aminopropyl)-polypropylene-glycol-block-polyethylene-glycol-block-polypropylene-glycol (Jeffamine® ED-900) (2.70 gr, 3.0 mmol) was added to the solution of 41 obtained above, followed by PyBOP (0.52 gr, 1.0 mmol). The reaction mixture was stirred at room temperature for 2 h, then another portion of PyBOP (0.52 gr., 1.0 mmol) was added and the stirring was continued for overnight. DMF was removed by rotary evaporation. Methanol (5 ml) was added to the reaction mixture and the resulting solution was poured into ethyl acetate (100 ml). The white precipitate was filtered and dried under reduced pressure (2.5 gr, 72% yield).

### iii. Synthesis of di-(mono amino β-CD)-Glu-SA-Jeffamine-FA 43

42 (1.0 mmol) and folic acid (FA, 0.882 gr., 2.0 mmol) were dissolved in anhydrous DMSO (20 ml). PyBOP (0.52 gr, 1.0 mmol) was added and the reaction mixture was stirred at room temperature for 2 h, then another portion of PyBOP (0.52 gr., 1.0 mmol) was added and the stirring was continued for overnight. The reaction mixture was poured into diethyl ether (250 ml). The oily orange precipitate was separated from the solution, dissolved in water (10 ml) and centrifuged to remove insoluble traces. The supernatant was dialyzed in Spectra/Por CE tubing (MW cutoff 2000) against distilled water (3×1000 mL). The dyalizate was lyophilized and the residue dried in vacuo over P₂O₅. The yield is 85%.

### Example 3

### Synthesis of (mono amino β-CD)₂-Glu-Glu-Jeffamine-folic acid derivative 45

The title derivative was synthesized starting from coupling the carboxy-protected CD-glutamic acid derivative 12 with the amino-protected CD-glutamic acid derivative 16 using HOBT and DCC in DMF to obtain the protected dipeptide Glu-Glu containing two CD residues 33 shown in Scheme 12. Then, the CD-containing homo dipeptide 34 was obtained by removing the N-protecting Boc group and the benzyl group from compound 33 using TFA and NaOH, as described in WO 2007/072481 and shown in Scheme 12.

### i. Synthesis of (mono amino β-CD)₂-Glu-Glu-Jeffamine 44

O,O′-bis(2-aminopropyl)-polypropylene-glycol-block-polyethylene-glycol-block-polypropylene-glycol (Jeffamine® ED-900) (2.70 gr, 3.0 mmol) and 34 (1.0 mmol) were dissolved in DMF (10 ml), followed by the addition of PyBOP (0.52 gr, 1.0 mmol). The reaction mixture was stirred at room temperature for 2 h, then another portion of PyBOP (0.52 gr., 1.0 mmol) was added and the stirring was continued for overnight. DMF was removed by rotary evaporation. Methanol (5 ml) was added to the reaction mixture and the resulting solution was poured into ethyl acetate (100 ml). The white precipitate was filtered and dried under reduced pressure (65% yield).

### ii. Synthesis of (mono amino β-CD)₂-Glu-Glu-Jeffamine-folic acid derivative 45

Derivative 44 (1.0 mmol) obtained above and folic acid (FA, 0.882 gr., 2.0 mmol) were dissolved in anhydrous DMSO (20 ml). PyBOP (0.52 gr, 1.0 mmol) was added and the reaction mixture was stirred at room temperature for 2 h, then another portion of PyBOP (0.52 gr., 1.0 mmol) was added and the stirring was continued for overnight. The reaction mixture was poured into diethyl ether (250 ml). The oily orange precipitate was separated from the solution, dissolved in water (10 ml) and centrifuged to remove insoluble traces. The supernatant was dialyzed in Spectra/Por CE tubing (MW cutoff 2000) against distilled water (3×1000 mL). The dyalizate was lyophilized and the residue dried in vacuo over P₂O₅. The yield is 80%.

### Example 4

### Synthesis of tri-(mono amino β-CD)-Glu-Glu-SA-Jeffamine-FA 48

### i. Synthesis of tri-(mono amino β-CD)-Glu-Glu 36

derivatives 31 (1.0 mmol), 16 (1.0 mmol), HOBT (2.0 mmol) and DCC (2.0 mmol) were dissolved in DMF (10 ml) and stirred at 25° C. for 3 days. The precipitate was filtered and the DMF was removed by evaporation under reduced pressure. The residue was triturated with hot acetone (100 ml). The precipitate was filtered and dried under vacuum.

The dried N-protected product was dissolved in TFA (10 ml) and CH₂Cl₂ (10 ml) and the mixture was stirred at 25° C. for 5 h. The solvent was removed by evaporation under reduced pressure (<25° C.) and the residue was poured into diethyl ether (200 ml). The white precipitate was filtered and dried under vacuum (65% yield). TLC analysis of 36 performed on silica plates (EtOAc:2-propanol:conc. NH₄OH:water-7:7:5:4) showed one major spot (R_{f}=0.02).

### ii. Synthesis of tri-(mono amino β-CD)-Glu-Glu-SA 46

Derivative 36 (1.0 mmol) and DMAP (0.12 gr, 1.0 mmol) were dissolved in DMF (5 ml). Succinic anydride (0.10 gr, 1.0 mmol) was added and the reaction mixture was stirred at 25° C. for 5 h.

### iii. Synthesis of tri-(mono amino β-CD)-Glu-Glu-SA-Jeffamine 47

O,O′-bis(2-aminopropyl)-polypropylene-glycol-block-polyethylene-glycol-block-polypropylene-glycol (Jeffamine® ED-900) (2.70 gr, 3.0 mmol) was added to the 47 solution obtained above, followed by PyBOP (0.52 gr, 1.0 mmol). The reaction mixture was stirred at room temperature for 2 h, then another portion of PyBOP (0.52 gr., 1.0 mmol) was added and the stirring was continued for overnight. DMF was removed by rotary evaporation. Methanol (5 ml) was added to the reaction mixture and the resulting solution was poured into ethyl acetate (100 ml). The white precipitate was filtered and dried under reduced pressure (76% yield).

### iv. Synthesis of tri-(mono amino β-CD)-Glu-Glu-SA-Jeffamine-FA 48

derivative 48 (1.0 mmol) and folic acid (FA, 0.882 gr., 2.0 mmol) were dissolved in anhydrous DMSO (20 ml). PyBOP (0.52 gr, 1.0 mmol) was added and the reaction mixture was stirred at room temperature for 2 h, then another portion of PyBOP (0.52 gr., 1.0 mmol) was added and the stirring was continued for overnight. The reaction mixture was poured into diethyl ether (250 ml). The oily orange precipitate was separated from the solution, dissolved in water (10 ml) and centrifuged to remove insoluble traces. The supernatant was dialyzed in Spectra/Por CE tubing (MW cutoff 3500) against distilled water (3×1000 mL). The dyalizate was lyophilized and the residue dried in vacuo over P₂O₅. The yield is 92%.

### Example 5

### Preparation of CD-Containing Peptides 35 by Grafting Native or Modified Cyclodextrins onto Peptides

A general procedure for the grafting of native or mono amino-CD or mono carboxy-CD onto a peptide having an amino acid residue with a —COOH or —COO⁻ or —NH₂ or —SH functional side group is depicted in Scheme 13.

For the preparation of a CD-containing peptide comprising glutamic acid and/or aspartic acid residues, a N-Boc-peptide of glutamic acid and/or aspartic acid, or a peptide-benzyl ester of glutamic acid and/or aspartic acid, or unprotected such peptide, HOBT and/or DMAP and DCC (or EDC or PyBOP) are dissolved in DMF (or DMSO or H₂O) and stirred at 25° C. for 1 h. A native or modified CD, e.g., β-CD or compound 4 or 5 or carboxy-CD or CD-NHCOCH₂CH₂COOH, is added and the stirring is continued for 48 h at 25° C. The precipitate is filtered and the solvent is removed by evaporation under reduced pressure. The residue is triturated with hot methanol. The precipitate is filtered and dried under vacuum to obtain the desired CD-containing polypeptide.

This procedure was applied in the grafting reaction of mono amino-CD onto poly-L-aspartic acid sodium salt (Mw=5000-15000, 36-109 amino acids) or poly-L-glutamic acid (Mw=2000-15000, 16-119 amino acids) or poly-L-glutamic acid sodium salt (Mw=750-3000, 5-20 amino acids) or poly-D-glutamic sodium salt (Mw=2000-15000, 13-100 amino acids) using HOBT. DMAP and EDC in water.

### Example 6

### Synthesis of CD-polyAsp-Jeffamine-FA 50

### i. Synthesis of CD-polyAsp-Jeffamine 49

O,O′-bis(2-aminopropyl)-polypropylene-glycol-block-polyethylene-glycol-block-polypropylene-glycol (Jeffamine® ED-900) (2.70 gr, 3.0 mmol) and 35 (1.0 mmol) obtained according to Example 5, were dissolved in DMF (10 ml), followed by the addition of PyBOP (0.52 gr, 1.0 mmol). The reaction mixture was stirred at room temperature for 2 h, then another portion of PyBOP (0.52 gr., 1.0 mmol) was added and the stirring was continued for overnight. DMF was removed by rotary evaporation. Methanol (5 ml) was added to the reaction mixture and the resulting solution was poured into ethyl acetate (100 ml). The white precipitate was filtered and dried under reduced pressure (50% yield).

### ii. Synthesis of CD-polyAsp-Jeffamine-FA 50

Polymer 49 and folic acid (FA, 0.882 gr., 2.0 mmol) were dissolved in anhydrous DMSO (20 ml). PyBOP (0.52 LIT, 1.0 mmol) was added and the reaction mixture was stirred at room temperature for 2 h, then another portion of PyBOP (0.52 gr., 1.0 mmol) was added and the stirring is continued for overnight. The reaction mixture was poured into diethyl ether (250 ml). The oily orange precipitate was separated from the solution, dissolved in water (10 ml) and centrifuged to remove insoluble traces. The supernatant was dialyzed in Spectra/Por CE tubing (MW cutoff 10,000) against distilled water (3×1000 ml). The dyalizate was lyophilized and the residue dried in vacuo over P₂O₅. The yield is 60%.

### Example 7

### General Procedure for Encapsulation of Guest Molecules

For the encapsulation process, a guest molecule (e.g., thymol, vitamin E, β-estardiol, cholesterol, taxol, doxorubicin, methyl orange, ethyl orange, phenol, toluene) (0.03 mmol) and a CD-containing polymer (0.01 mmol) are completely dissolved in water or a mixture of ethanol and water (10%:90%) or methanol/water and stirred for 3 days at room temperature. After evaporating the ethanol/methanol from the stirred solution, the non encapsulated guest molecule is removed by filtration. The filtrate is again evaporated to remove water and dried in vacuum to give encapsulated guest CD-containing polymer complex (yield ˜90%).

### Example 8

### Binding Cyclodextrin Polymer to Folic Acid

For synthesis of conjugates comprising folic acid, the folic acid was first activated by esterification with the leaving group N-hydroxysuccinimide.

a) N-hydroxysuccinimide ester of folic acid (NHS-folate) is prepared by the following method:

Folic acid (4.41 g, 10 mmol) and triethylamine (2.5 ml) are dissolved in dry DMSO (100 ml). N-hydroxysuccinimide (2.30 g, 20 mmol) and DCC (4.12 g, 20 mmol) are added and the mixture is stirred at room temperature for 24 h. The by-product dicyclohexylurea is removed by filtration and the DMSO solution is concentrated under reduced pressure at <60° C. The NHS-folate product is precipitated in diethyl ether, washed several times with anhydrous ether and dried under vacuum affording 4.5 g (84% yield) as a yellow powder.

b) A CD-containing polymer conjugated to folic acid is prepared by the following method:

NHS-folate (1.0 mmol) is dissolved in DMSO (10 ml). A CD-containing polymer (10 mmol) is added and stirred at room temperature for overnight. The mixture is poured into acetone (200 ml), Filtered, washed several times with methanol and dried under vacuum.

### Example 9

### Synthesis of [(mono amino β-CD)-poly-Glu]-PEG₃₃₅₀-Folic acid 38

For the synthesis of the title conjugate, the folic acid was first activated by esterification with the leaving group N-hydroxysuccinimide, as described in Example 9 above.

a) N-hydroxysuccinimide ester of folic acid (NHS-folate) was prepared by dissolving folic acid (0.441 g, 1 mmol) and triethylamine (0.25 ml) in dry DMSO (20 ml). NHS (0.165 g, 1.1 mmol) and DCC (0.227 g, 1.1 mmol) were added and the mixture was stirred at room temperature for 24 h. The by-product dicyclohexylurea (DCU) was removed by filtration and the DMSO solution of NHS-folate was kept at −20° C.

b) Polyethyleneglycol diamine(H₂N-PEG-NH₂, Mw=3350) conjugated to folic acid (H₂N-PEG-NH-folic acid) was prepared by the following method: 2 ml of the DMSO solution of NHS-folate (54 mg, ˜0.1 mmol) obtained in (a) was added to a solution of polyethyleneglycol diamine (335 mg, 0.1 mmol) in 3 ml DMSO. The reaction mixture was stirred at room temperature for 24 h. The resulting solution of H₂N-PEG-NH-folic acid was used in the next step (c) without isolation or purification of the intermediate product.

c) Coupling of H₂N-PEG-NH-folic acid with (mono amino β-CD)₅₀-polyGlu 37, namely 50% mono-amino β-CD-grafted polyGlu, was carried out as follow: 37 (140 mg), HOBT (41 mg, 0.3 mmol) and DMAP (36 mg, 0.3 mmol) were dissolved in the DMSO solution of H₂N-PEG-NH-folic acid obtained in (b). EDC (60 mg, 0.3 mmol) was added and the solution was stirred at 25° C. for 48 h. The reaction mixture was poured into acetone (100 ml), and the precipitate was filtered and dried under vacuum yielding product 38 as a pale-yellow powder.

All products were analyzed by HPLC chromatography and NMR spectroscopy.

### Example 10

### Synthesis of Compounds 51, 52, 53 and 54

Compound 51 (mono-6-deoxy-6-(4-carboxy-4-amino butyrate)-β-cyclodextrin), wherein the cyclodextrin is directly bound via an enteric bond to the free carboxylic functional side group of the glutamic acid through the CD's hydroxy group (OH) at position 6, is prepared starting with the diprotected amino acid N-carboxybenzyl-glutamic acid α-benzyl ester. The ester bond between the CD and the amino acid is kept intact during deprotection by using catalytic hydrogeneation (H₂/C/Pd in methanol/water) to remove the protecting groups.

### (i) Synthesis of (N-carboxybenzyl-glutamic acid α-benzyl ester)-β-cyclodextrin

N-carboxybenzyl-glutamic acid α-benzyl ester (1.0 mmol), HOBT (2.0 mmol), DMAP (2.0 mmol) and EDC (2.0 mmol), are added to DMF (10 ml) and the reaction mixture is stirred at 25° C. for 2 h. Dry β-cyclodextrin (2.0 mmol) is added in one portion and the stirring is continued for 48 h at 25° C. The solvent is removed by evaporation under reduced pressure, and the oily residue is dissolved in hot water and purified by reversed-phase chromatography (eluent: from 5% methanol/95% water to 50% methanol/50% water). The product is recrystallized from hot water (73% yield based on amino acid).

### (ii) Deprotection

The N-carboxybenzyl-α-benzyl ester glutamic acid ester of β-CD (1.0 mmol) is dissolved in water/methanol (50 ml, 1:1) by stirring at 25° C. for 1 h. Pd/C powder (0.5 gr) is added under nitrogen atmosphere. Excess of hydrogen (H₂) is added (2 atm) with stirring at 25° C. for 24 h. The solvent is removed by evaporation under reduced pressure, and the residue is dissolved in water (2 ml) and poured into acetone 1 (250 ml). The white precipitate is filtered and dried under reduced pressure (95% yield).

Compounds 52, 53 and 54 (mono-6-deoxy-6-(3-carboxy-3-amino propionate)-β-cyclodextrin, mono-6-deoxy-6-(butyroylamino ethoxy)-β-cyclodextrin and mono-6-deoxy-6-(propionylamino ethoxy)-β-cyclodextrin, respectively) are prepared in a similar manner, starting with the corresponding di-protected anibo acid (e.g., N-carboxybenzyl-aspartic acid α-benzyl ester), and using the unique combination of EDC-HOBT-DMAP as coupling reagents and DMF as the solvent. Selective deprotection of the carboxy and amino groups while keeping the esteric bond to CD intact was made possible by employing protecting groups comprising benzyl, and using catalytic hydrogeneation (H₂/C/Pd in methanol/water) to remove the protecting groups.

### Example 11

### Synthesis of the conjugates di-CD-Glu-PEG₃₃₅₀-FA-RhB 55, tri-CD-Glu-Glu-PEG₃₃₅₀-FA-RhB 56 and CD-polyGlu-PEG₃₃₅₀-FA-RhB 57

Conjugates of di-CD-Glu-PEG₃₃₅₀-FA, tri-CD-Glu-Glu-PEG₃₃₅₀-FA, and CD-polyGlu-PEG₃₃₅₀-FA encapsulating the fluorescence compound rhodamine-B (RhB), were prepared by mixing di-CD-Glu-PEG₃₃₅₀-FA, tri-CD-Glu-Glu-PEG₃₃₅₀-FA, and CD-polyGlu-PEG₃₃₅₀-FA with RhB under condition described in Example 7 above.

### Example 12

### In Vitro Binding of Conjugates 55, 56 and 57

In this study the capacity of conjugates 55, 56 and 57 encapsulating the fluorescent marker rhodamine-B (RhB) to bind to human nasopharyngeal KB cancer cells (herein KB cells), which overexpress the folate receptor (FR), was tested.

KB cancer cells were cultured as described in Materials and Methods, and seeded on both Black and transparent 96 well plates for fluorescence counting and fluorescent microscopy. Each of the above conjugates were loaded with 0.1 mM RhB and diluted into fresh medium to the final concentrations 0.1-100 μM (triplicate preparations were prepared). As controls, mixtures of non-encapsulated RhB and free di-CD-GluPEG₃₃₅₀-FA, tri-CD-Glu-Glu, and CD-polyGlu, PEG₃₃₅ and biorecognition moiety FA, each at a concentration of 0.1 mM, were used. Twenty-four hours after seeding, the old medium was replaced with the conjugate-containing medium and cell were incubated for 30 minutes at 37° C. The medium was then washed 3 times with PBS 1 X, and fluorescence associated with the cells in both plates was counted using Analyst HT (Ex 525 nm, Dc 560 nm, Em 595 nm). The net fluorescence was calculated by subtracting the averaged background fluorescence form the fluorescence of the conjugate-treated cells. The transparent plate was further analyzed by fluorescence microscopy.

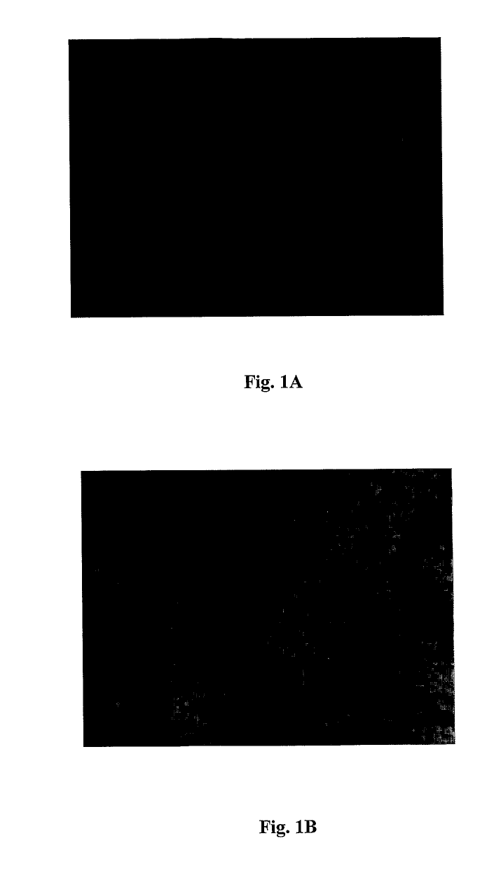

As shown in FIGS. 1A-1B, the fluorescence associated with folate-receptor over-expressing KB cancer cells incubated with the RhB-encapsulating di-CD-Glu-PEG₃₃₅₀-FA, (FIG. 1B), was by far more intense than the fluorescence obtained from control cells (FIG. 1A). In fact, the fluorescence of cells treated with the RhB-loaded conjugate was 780% higher relative to control.

Fluorescence counting resulted in 4,000,000 RFU for cells treated with conjugates 55 and 56, and 12,000,000 RFU for cell treated with conjugate 57, compared to ˜2,000,000 RFU obtained for the corresponding controls. These date indicate that encapsulating and targeting the delivery of an active agent using the conjugates of the invention is far more effective compared to non encapsulated and non targeted delivery of same.

In the following pages, the Schemes 1-16 mentioned above are depicted. In the schemes, n in the cyclodextrin ring means a value of 6, 7 or 8.

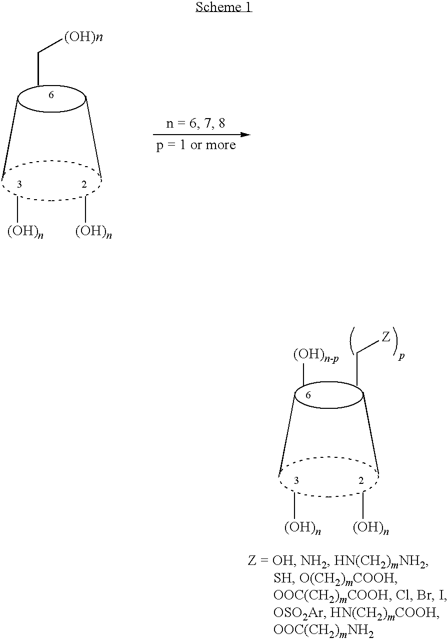

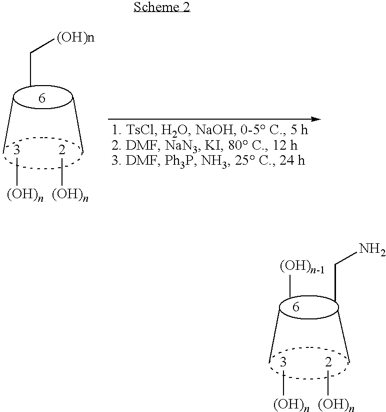

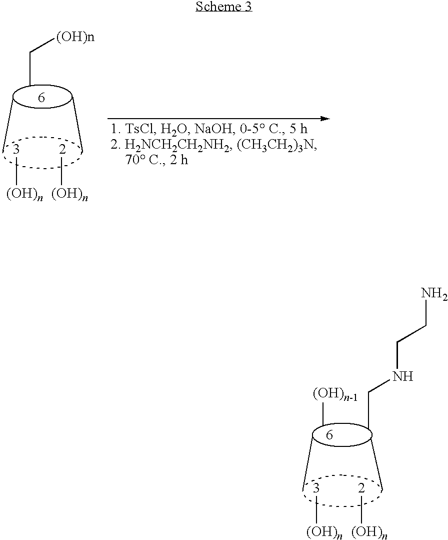

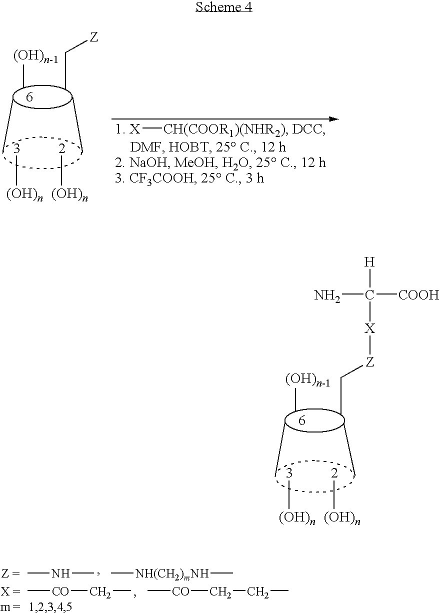

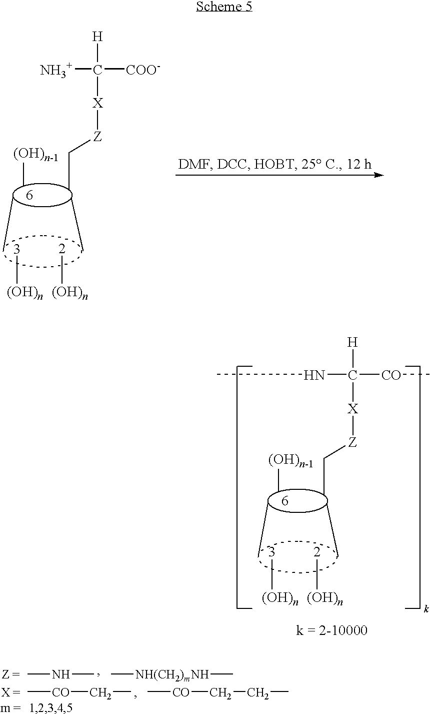

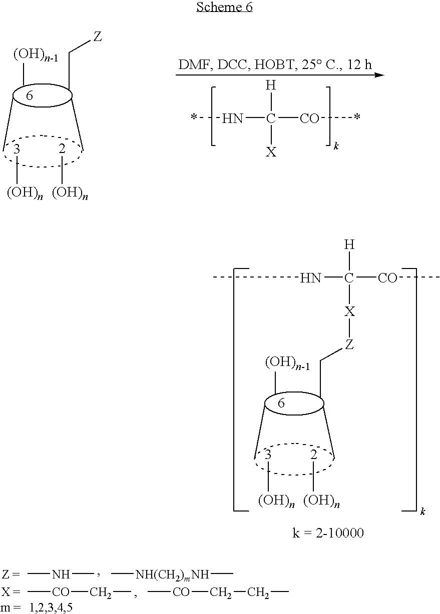

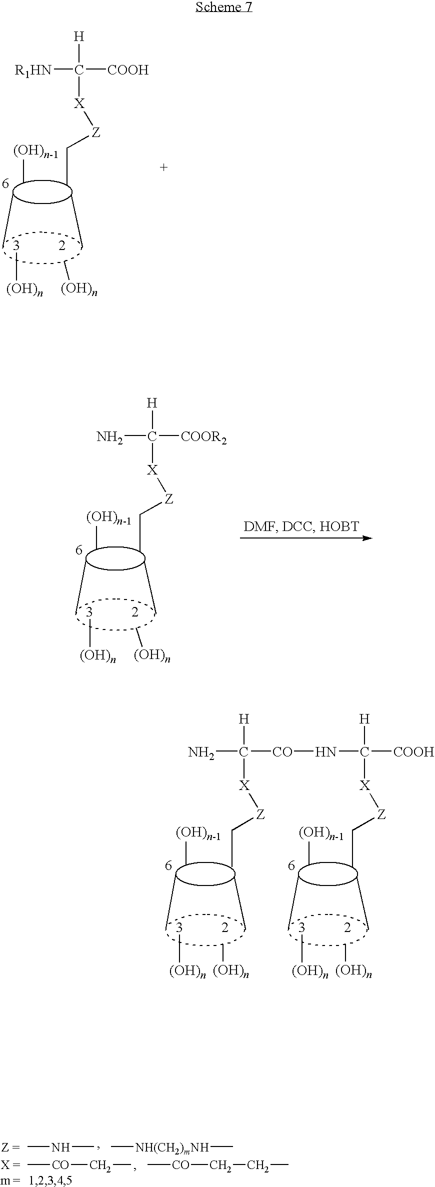

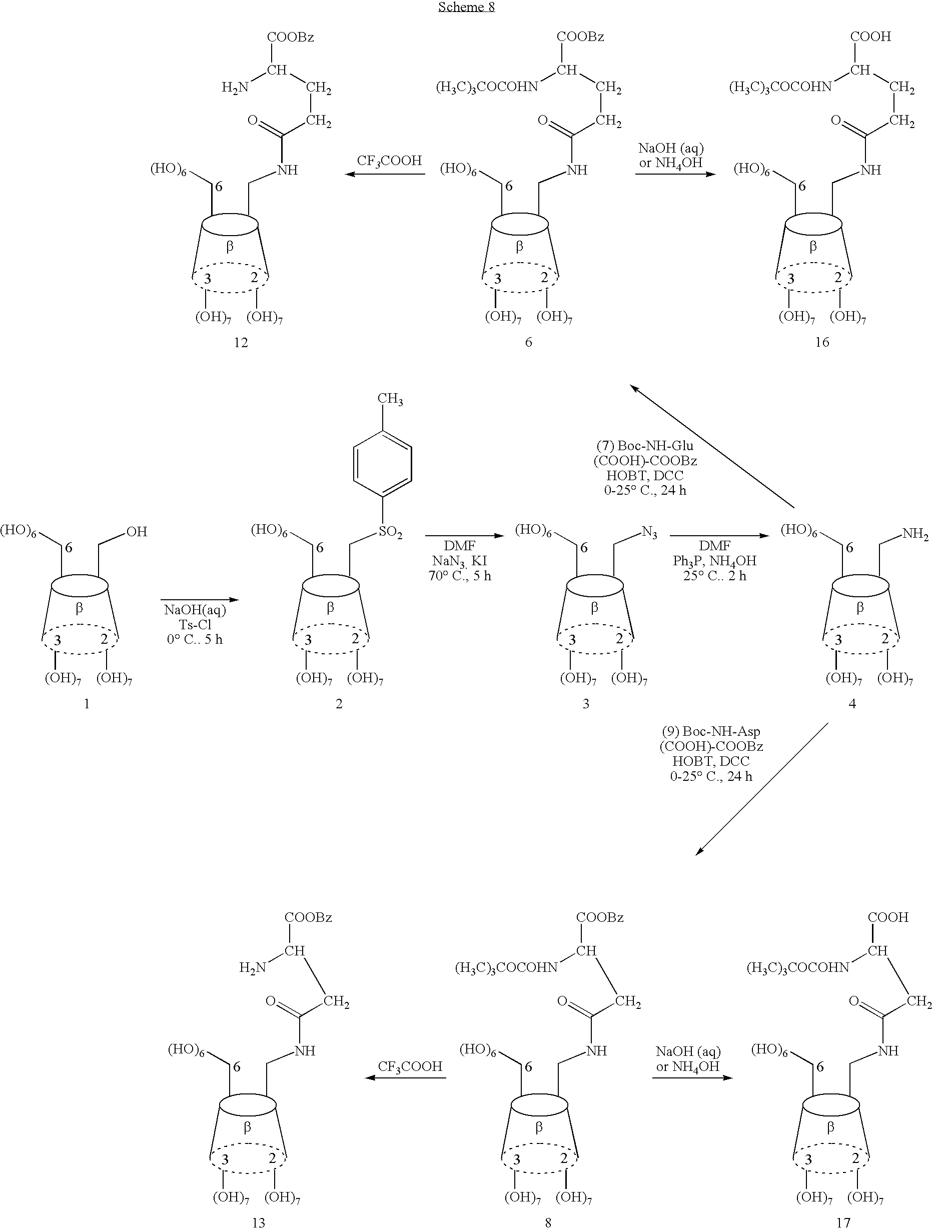

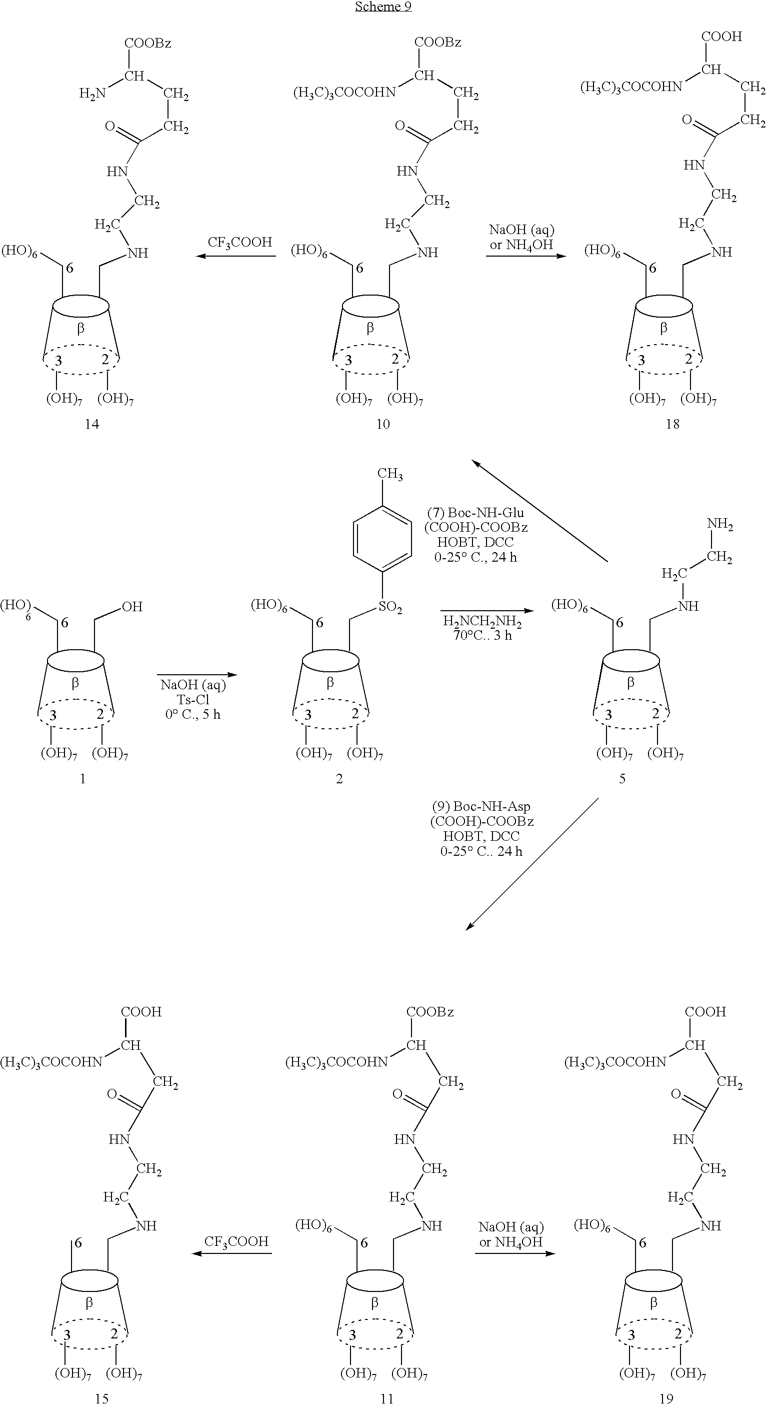

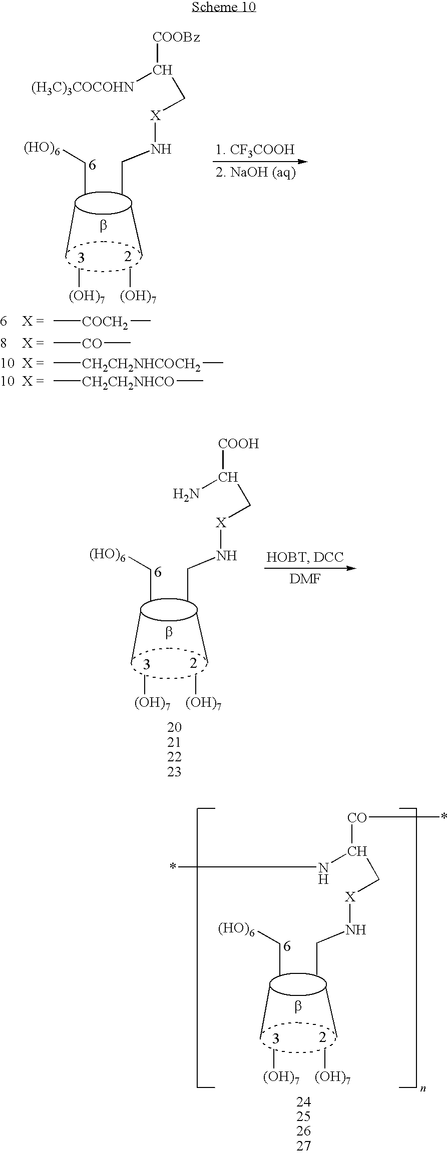

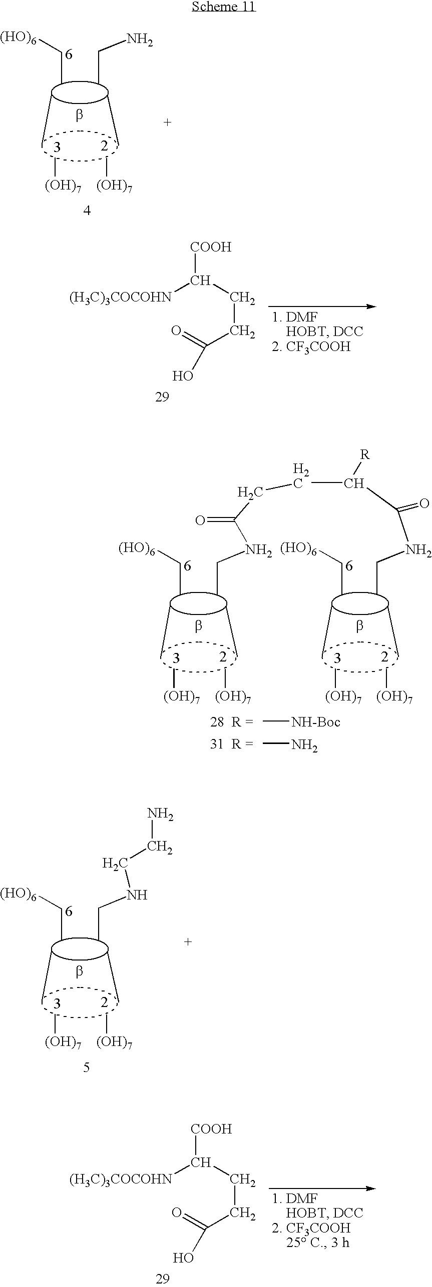

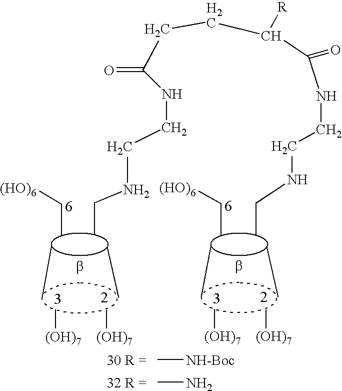

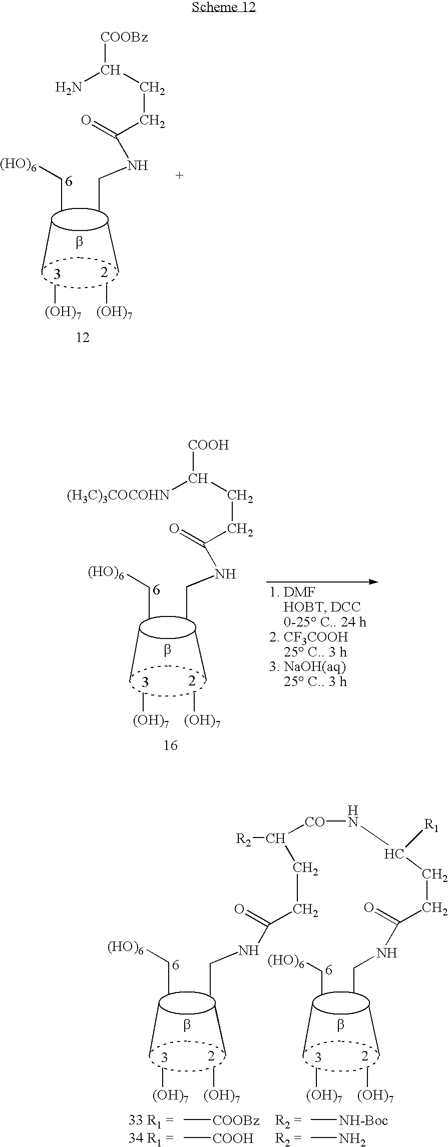

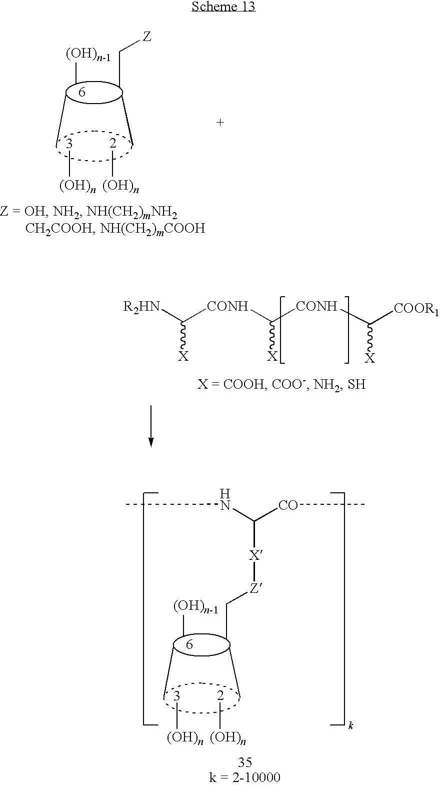

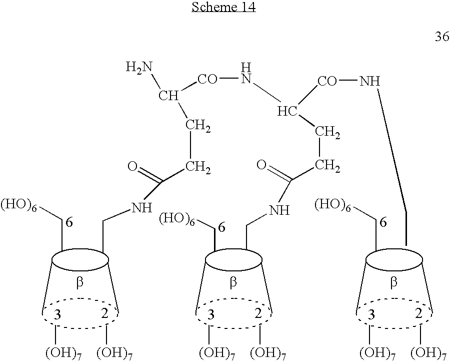

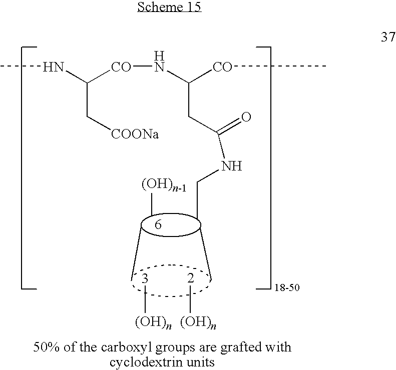

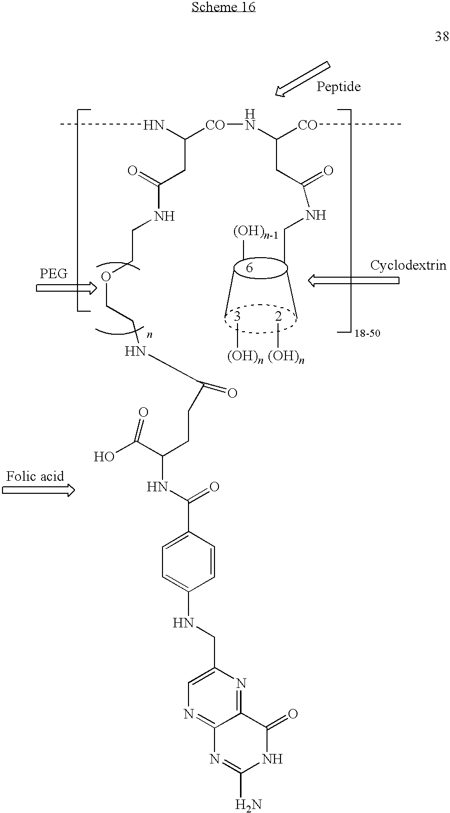

### REFERENCES

Barse B., Kaul P., Banerjee A., Kaul, C. L. and Banerjee. U. C., 2003. “Cyclodextrins: Emerging applications” Chimica Oggi, 21: 48-54.
Li J. and Liu D. 2003, “Progress of the Application of beta-Cyclodextrin and Its Derivatives in Analytical Chemistry” Physical Testing and Chemical Analysis Part B Chemical Analysis, 39(6):372-376.
Parrot-Lopez H., Djedaini F., Perly B., Coleman A. W., Galons, H. and Miocque M. 1990a. Tetrahedron Lett., 31: 1999-2002.
Parrot-Lopez H., Galons H., Coleman A. W., Djedaini F., Keller N. and Perly B. 1990b. Tetrahedron Asymmetry, 1: 367-370.
Parrot-Lopez H., Galons H., Dupas S., Miocque M. and Tsoucaris G. 1990c Bull. Soc. Chim. Fr., 127: 568-571.
Takahashi K., Ohtasuka Y., Nakada S. and Hattori. K. 1991 J. Incl. Phenom., 10: 63-68.

1. An active agent-cyclodextrin containing polymer-biorecognition molecule conjugate, wherein: (i) said cyclodextrin (CD) containing polymer comprises one or more CD residues, said polymer is selected from a peptide, a polypeptide, an oligonucleotide or a polynucleotide, the peptide or polypeptide comprises at least one amino acid residue containing a functional side group and at least one of the CD residues is linked covalently to said functional side group or to the sugar moiety of a nucleotide residue of said oligonucleotide or polynucleotide; (ii) said biorecognition molecule is covalently bonded directly or via a spacer to the polymer backbone of the CD-containing polymer; and (iii) said active agent is non-covalently encapsulated within the cavity of the cyclodextrin residues and/or entrapped within the polymer matrix of the CD-polymer.

2. The conjugate according to claim 1, comprising one or more cyclodextrin residues selected from α-, β-, γ-cyclodextrin, a combination thereof, derivatives, analogs or isomers thereof, wherein at least one of the cyclodextrin residues is covalently linked to a functional side group of an amino acid residue of an all-L, all-D or L,D-peptide or polypeptide, in which the amino acids may be natural amino acids, non-natural amino acids or chemically modified amino acids containing a functional side group.

3. The conjugate according to claim 2, wherein said at least one amino acid containing a functional side group is lysine, aspartic acid, glutamic acid, cysteine, serine, threonine, tyrosine or histidine.

4. The conjugate according to claim 1, wherein said peptide is an oligopeptide of 2-20.

5. The conjugate according to claim 4, wherein said oligopeptide is the dipeptide-Glu-Glu, Asp-Asp, Lys-Lys or Cys-Cys.

6. The conjugate according to claim 1, wherein said polypeptide or protein has 21 to 10,000.

7. The conjugate according to claim 6, wherein the polypeptide is a homopolypeptide of an amino acid having a functional side group such as polylysine, polyglutamic acid, polyaspartic acid, polycysteine, polyserine, polythreonine or polytyrosine.

8. The conjugate according to claim 1, wherein said biorecognition molecule is a peptide, a protein, a lipid, a carbohydrate, an oligonucleotide, a polynucleotide, or an organic molecule which binds to a target site.

9. The conjugate according to claim 8, wherein the biorecognition molecule is a protein selected from the group consisting of antibodies, antigens, hormones, cytokines, enzymes, and receptors.

10. The conjugate according to claim 9, wherein said antibodies include monoclonal and polyclonal antibodies, fragments such as the Fab and Fc fragments, chimeric and humanized antibodies and derivatives thereof.

11. The conjugate according to claim 10, wherein said antibody is a chimeric or humanized anticancer monoclonal antibody.

12. The conjugate according to claim 1, wherein said active agent is a compound that has therapeutic, inhibitory, antimetabolic, or preventive activity toward a disease or it is inhibitory or toxic toward any disease causing agent or it is a label or marker, said active agent is selected from prodrugs, anticancer drugs, antineoplastic drugs, antifungal drugs, antibacterial drugs, antiviral drugs, cardiac drugs, neurological drugs, and drugs of abuse, or said active agent is a fluorescent label.

13-14. (canceled)

15. The conjugate according to claim 1, wherein said biorecognition molecule targets to cancer cells and said active agent is (i) an anticancer drug; or (ii) a fluorescent marker.

16. The conjugate according to claim 15 wherein said biorecognition molecule is an anticancer monoclonal antibody or folic acid and (i) said anticancer drug is doxorubicin or paclitaxel; or (ii) said fluorescent marker is rhodamine B.

17-18. (canceled)

19. The conjugate according to claim 1, wherein the biorecognition molecule is linked to the polymer backbone of the CD-containing polymer via a linking group selected from a polyether or a polyether amine residue.

20. The conjugate according to claim 19, wherein said linking group is polyethylene glycol of MW 10-50,000 (PEG_{10-50,000}), O,O′-bis(2-aminopropyl)polypropylene glycol or O,O′-bis(2-aminopropyl)polypropylene glycol-block-polyethylene glycol-block-polypropylene glycol.

21. A pharmaceutical composition comprising an active agent-cyclodextrin containing polymer-biorecognition molecule conjugate as defined in claim 1.

22. A cyclodextrin containing polymer-biorecognition molecule compound, wherein: (i) said cyclodextrin (CD) containing polymer comprises one or more CD residues, said polymer is selected from a peptide, a polypeptide, an oligonucleotide or a polynucleotide, the peptide or polypeptide comprises at least one amino acid residue containing a functional side group and at least one of the CD residues is linked covalently to said functional side group or to the sugar moiety of a nucleotide residue of said oligonucleotide or polynucleotide; and (ii) said biorecognition molecule is covalently bonded directly or via a spacer to the polymer backbone of the CD-containing polymer.

23. The conjugate according to claim 20, wherein said linking group is polyethylene glycol of MW 3350 (PEG₃₃₅₀).
